(19) United States
(12) Patent Application Publication
(10) Pub. No.: US 2012/0059179 A1
(43) Pub. Date: Mar. 8, 2012

(54) PALLADIUM-CATALYZED ORTHO-FLUORINATION

(75) Inventor: Jin-Quan Yu, San Diego, CA (US)

(73) Assignee: The Scripps Research Insitiute, La Jolla, CA (US)

(21) Appl. No.: 13/262,593

(22) PCT Filed: Apr. 2, 2010
(86) PCT No.: PCT/US10/29775

§ 371 Date: Nov. 22, 2011

Related U.S. Application Data
(60) Provisional application No. 61/166,599, filed on Apr. 3, 2009.

(51) Int. Cl. C07C 209/74; C07C 253/00; C07C 67/30; C07C 247/10

(52) U.S. Cl. 552/10; 564/384; 558/350; 560/105; 568/436

(57) ABSTRACT

A new method of ortho-fluorination where an aryl C—H bond is directly replaced by an aryl C-F bond in a palladium-catalyzed reaction is provided. The method includes the ortho-fluorination of a triflamide protected benzylamine, a palladium catalyst, such as Pd(OTf)₂, a fluorinating reagent such as N-fluoro-2,4,6-trimethylpyridinium triflate, and a ligand to promote the reaction such as N-methylpyrrolidinone (NMP).

### CROSS REFERENCE TO RELATED APPLICATIONS

This application claims priority to U.S. provisional application No. 61/166,599, filed Apr. 3, 2009, which is incorporated by reference in its entirety.

### FIELD OF THE INVENTION

Embodiments of the invention are directed to methods for regioselectivity replacing C—H bonds directly by C—F bonds. Compositions synthesized by these methods comprise fluorinated molecules.

### BACKGROUND

Aryl fluoride (ArF) moieties have long been recognized as privileged pharmacophores; not only are they isosteric to the parent, non-fluorinated arenes, but they exhibit improved lipophilicity as well as inertness to metabolic transformations (Shimizu, M.; Hiyama, T. Angew. Chem., Int. Ed. 2005, 44, 214; Tredwell, M.; Gouverneur, V. Org. Biomol. Chem. 2006, 4, 26; Mailer, K.; Faeh, C.; Diederich, F. Science 2007, 317, 1881). Therefore, development of new methods for the introduction of fluorines into arenes is a significant task. The ortho-lithiation/fluorination protocol with a fluorine source reported by Snieckus and Davis represents an approach for regioselective fluorination of arenes (Snieckus, V.; et al., Tetrahedron Lett. 1994, 35, 3465). In light of the remarkable success of the Pd(0)-catalyzed carbon heteroatom formation processes, especially the Buchwald-Hartwig amination reaction (Handbook of Organopalladium Chemistry for Organic Synthesis; Negishi, E. I., Ed.; Wiley-Interscience: New York, 2002), Pd(0)-catalyzed displacement of halides by fluoride would appear to be the most viable approach. However, reductive elimination of fluoride from Pd(II) species is notoriously challenging owing to the high strength of the Pd—F bond and has been a formidable problem to overcome.

### SUMMARY

This Summary is provided to present a summary of the invention to briefly indicate the nature and substance of the invention. It is submitted with the understanding that it will not be used to interpret or limit the scope or meaning of the claims.

Methods for efficient ortho-fluorination of an arene using a palladium(II) catalyst, a fluorinating reagent, and a promoter are provided herewith. This method provides for the direct addition of one or two fluorine groups ortho to N-protected aminomethylarenes.

In one embodiment, methods for efficient ortho-fluorination comprise a palladium catalyst, such as, for example, Pd(OTf)₂, a triflamide, for example, N-fluoro-2,4,6-trimethylpyridinium triflate as the fluorine source; and, N-methylpyrrolidinone (NMP) as the ligand to promote the reaction.

The protecting group on the aminomethylarenes can be readily displaced by a wide range of heteroatom and carbon nucleophiles, thereby affording this fluorination protocol excellent versatility for synthetic applications.

Other aspects are described infra.

### DETAILED DESCRIPTION

Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

### Definitions

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “alkyl” refers to an optionally substituted, saturated, straight or branched hydrocarbon having from about 1 to about 20 carbon atoms (and all combinations and subcombinations of ranges and specific numbers of carbon atoms therein), preferably with from about 1 to about 8 carbon atoms, more preferably with from about 1 to about 6 carbon atoms, even more preferably with from about 1 to about 4 carbon atoms, still more preferably with from about 1 to about 3 carbon atoms, with 1 carbon being especially preferred in some embodiments. For example, the term “C₁₋₆ alkyl” means a straight or branched alkyl containing at least 1 and at most 6 carbon atoms. In some embodiments, the alkyl is optionally substituted. For example, 1 or more of the hydrogen atoms on the alkyl group, preferably from 1 to about 6, more preferably about 1 to about 3 of hydrogen atoms on the alkyl group are substituted with a F, Cl, Br, NH₂, NO₂, N₃, CN, COOH, OH, etc. Alkyl groups include, but are not limited to, methyl, ethyl, n-propyl, isopropyl, n-butyl, isobutyl, sec-butyl, t-butyl, n-pentyl, isopentyl, neopentyl, n-hexyl, isohexyl, 3-methylpentyl, 2,2-dimethylbutyl, and 2,3-dimethylbutyl.

As used herein, the term “cycloalkyl” refers to an optionally substituted, mono-, di-, tri-, or other multicyclic alicyclic ring system having from about 3 to about 20 carbon atoms (and all combinations and subcombinations of ranges and specific numbers of carbon atoms therein). In some preferred embodiments, the cycloalkyl groups have from about 3 to about 10 carbon atoms, more preferably from about 3 to about 8 carbon atoms, with from about 3 to about 6 carbon atoms being preferred. For example, the term “C₃₋₆ cycloalkyl” means a mono- or bicyclic saturated ring structure containing at least 3 and at most 6 carbon atoms. Multi-ring structures may be bridged or fused ring structures, wherein the additional groups fused or bridged to the cycloalkyl ring may include optionally substituted cycloalkyl, aryl, heterocycloalkyl, or heteroaryl rings. Exemplary cycloalkyl groups include, but are not limited to, cyclopropyl, cyclobutyl, cyclopentyl, cyclohexyl, cyclooctyl, adamantyl, 2-[4-i sopropyl-1-methyl-7-oxa-bicyclo[2.2. 1]-heptanyl], and 2- [1,2,3,4-tetrahydro-naphthalenyl].

As used herein, the term “alkylcycloalkyl” refers to an optionally substituted ring system comprising a cycloalkyl group substituted with one or more alkyl substituents, wherein cycloalkyl and alkyl are each as previously defined. Exemplary alkylcycloalkyl groups include 2-methylcyclohexyl, 3,3-dimethylcyclopentyl, trans-2,3-dimethylcyclooctyl, and 4-methyldecahydronaphthalenyl.

As used herein, the term “alkenyl” as a group or a part of a group refers to an optionally substituted straight or branched hydrocarbon chain containing the specified number of carbon atoms and containing at least one double bond. For example, the term “C₂₋₆ alkenyl” means a straight or branched alkenyl containing at least 2 and at most 6 carbon atoms and containing at least one double bond. Multiple double bonds may be adjacent (═C═), conjugated (═C—C═), or are non-adjacent and non-conjugated. In particular, multiple double bonds are conjugated, or are non-adjacent and non-conjugated. It will be appreciated that in groups of the form —O—C₂₋₆ alkenyl, the double bond is preferably not adjacent to the oxygen. Preferably, the alkenyl groups of the invention may be optionally substituted and have from about 2 to about 10 carbon atoms (and all combinations and subcombinations of ranges and specific numbers of carbon atoms therein), more preferably 2 to 6 carbon atoms. Examples of “alkenyl” as used herein include, but are not limited to, ethenyl, 2-propenyl, 3-butenyl, 2-butenyl, 2-pentenyl, 3-pentenyl, 3-methyl-2-butenyl, 3-methylbut-2-enyl, 3-hexenyl and 1,1-dimethylbut-2-enyl.

The term “alkynyl” as used herein as a group or a part of a group refers to an optionally substituted straight or branched hydrocarbon chain containing the specified number of carbon atoms and containing at least one triple bond. For example, the term “C₂₋₆ alkynyl” means a straight or branched alkynyl containing at least 2, and at most 6, carbon atoms and containing at least one triple bond. Multiple triple bonds may be conjugated or non-conjugated. In particular, multiple triple bonds are non-conjugated. It will be appreciated that in groups of the form —O—C₂₋₆ alkynyl, the triple bond is preferably not adjacent to the oxygen. Preferably, the alkynyl groups of the invention may be optionally substituted and have from about 2 to about 10 (and all combinations and subcombinations of ranges and specific numbers of carbon atoms therein) carbon atoms, preferably 2 to 6 carbon atoms. Examples of “alkynyl” as used herein include, but are not limited to, ethynyl, 2-propynyl, 3-butynyl, 2-butynyl, 2-pentynyl, 3-pentynyl, 3-methyl-2-butynyl, 3-methylbut-2-ynyl, 3-hexynyl and 1,1-dimethylbut-2-ynyl.

As used herein, the term “aryl” refers to an optionally substituted, mono-, di-, tri-, or other multicyclic aromatic ring system having from about 6 to about 50 carbon atoms (and all combinations and subcombinations of ranges and specific numbers of carbon atoms therein), preferably with from about 6 to about 14 carbons, with about 6 to 10 carbon atoms being preferred. Non-limiting examples include, for example, phenyl, naphthyl, anthracenyl, and phenanthrenyl. Aryl may optionally be further fused to an aliphatic or aryl group or can be substituted with one or more substituents such as halogen (fluorine, chlorine and/or bromine), hydroxy, alkyl, alkoxy or aryloxy, amido, nitro, alkylenedioxy, alkylthio or arylthio, alkylsulfonyl, cyano, or primary, secondary or tertiary amino.

As used herein, the term “alkoxy” refers to an optionally substituted straight or branched chain alkyl-O— group wherein alkyl is as previously defined. For example, C₁₋₆ alkoxy means a straight or branched alkoxy containing at least 1, and at most 6, carbon atoms. Examples of “alkoxy” as used herein include, but are not limited to, methoxy, ethoxy, propoxy, prop-2-oxy, butoxy, but-2-oxy, 2-methylprop- 1 -oxy, 2-methylprop-2-oxy, pentoxy and hexyloxy. A C₁-₄ alkoxy group is preferred, for example methoxy, ethoxy, propoxy, prop-2-oxy, butoxy, but-2-oxy or 2-methylprop-2-oxy. In some preferred embodiments, the alkyl moieties of the alkoxy groups have from about 1 to about 4 carbon atoms. As used herein, the term “aryloxy” refers to an optionally substituted aryl-O-group wherein aryl is as previously defined. Exemplary aryloxy groups include, but are not limited to, phenoxy (phenyl-O—) and naphthoxy (naphthyl-O).

As used herein, the term “heteroaryl” refers to an optionally substituted aryl ring system wherein, in at least one of the rings, one or more of the carbon atom ring members is independently replaced by a heteroatom group selected from the group consisting of S, O, N, and NH, or NR wherein aryl is as previously defined and R is an optional substitutent as defined herein. Heteroaryl groups having a total of from about 5 to about 14 carbon atom ring members and heteroatom ring members (and all combinations and subcombinations of ranges and specific numbers of carbon and heteroatom ring members) are preferred. Heteroaryl groups having a total of from about 5 to about 10 carbon atom ring members and heteroatom ring members (and all combinations and subcombinations of ranges and specific numbers of carbon and heteroatom ring members) are more preferred. Exemplary heteroaryl groups include, but are not limited to, pyrryl, furyl, pyridyl, pyridine-N-oxide, 1,2,4-thiadiazolyl, pyrimidyl, thienyl, isothiazolyl, imidazolyl, tetrazolyl, pyrazinyl, pyrimidyl, quinolyl, isoquinolyl, thiophenyl, benzothienyl, isobenzofuryl, pyrazolyl, indolyl, purinyl, carbazolyl, benzimidazolyl, and isoxazolyl. Heteroaryl may be attached to the rest of the molecule via a carbon or a heteroatom.

As used herein, the term “heteroarylalkyl” refers to an optionally substituted ring system comprising an alkyl radical bearing a heteroaryl substituent, each as defined above, having from about 6 to about 50 carbon atoms (and all combinations and subcombinations of ranges and specific numbers of carbon atoms therein), with from about 6 to about 25 carbon atoms being preferred. Non-limiting examples include 2-(1H-pyrrol-3-yl)ethyl, 3-pyridylmethyl, 5-(2H-tetrazolyl)methyl, and 3-(pyrimidin-2-yl)-2-methylcyclopentanyl.

As used herein, the term “heterocycloalkyl,” “heterocyclic ring” and “heterocyclyl” each refer to an optionally substituted ring system composed of a cycloalkyl radical wherein, in at least one of the rings, one or more of the carbon atom ring members is independently replaced by a heteroatom group selected from the group consisting of O, S, N, and NH, or NR wherein cycloalkyl is as previously defined and R is an optional substituent as defined herein. Heterocycloalkyl ring systems having a total of from about 3 to about 14 carbon atom ring members and heteroatom ring members (and all combinations and subcombinations of ranges and specific numbers of carbon and heteroatom ring members) are preferred, more preferably from about 3 to about 10 ring atom members. In other preferred embodiments, the heterocyclic groups may be fused to one or more aromatic rings. In certain preferred embodiments, heterocycloalkyl moieties are attached via a ring carbon atom to the rest of the molecule. Exemplary heterocycloalkyl groups include, but are not limited to, azepanyl, tetrahydrofuranyl, hexahydropyrimidinyl, tetrahydrothienyl, piperidinyl, pyrrolidinyl, isoxazolidinyl, isothiazolidinyl, pyrazolidinyl, oxazolidinyl, thiazolidinyl, piperazinyl, 2-oxo-morpholinyl, morpholinyl, 2-oxo-piperidinyl, piperadinyl, decahydroquinolyl, octahydrochromenyl, octahydro-cyclopentapyranyl, 1,2,3,4,-tetrahydroquinolyl, 1,2,3,4-tetrahydroquinazolinyl, octahydro-[2]pyridinyl, decahydro-cyclooctafuranyl, 1,2,3,4-tetrahydroisoquinolyl, 2-oxo-imidazolidinyl, and imidazolidinyl. In some embodiments, two moieties attached to a heteroatom may be taken together to form a heterocycloalkyl ring. In certain of these embodiments, 1 or 2 of the heterocycloalkyl ring carbon atoms may be replaced by other moieties which contain either one (—O—, —S—, —N(R⁹)—) or two) (—N(R¹⁰)—C(═O)—, or —C(═O)—N(R¹⁰)—) ring replacement atoms. When a moiety containing one ring replacement atom replaces a ring carbon atom, the resultant ring, after replacement of a ring atom by the moiety, will contain the same number of ring atoms as the ring before ring atom replacement. When a moiety containing two ring replacement atoms replaces a ring carbon atom, the resultant ring after replacement will contain one more ring atom than the ring prior to replacement by the moiety. For example, when a piperidine ring has one of its ring carbon atoms replaced by —N(R¹⁰)—C(═O)—, the resultant ring is a 7-membered ring containing 2 ring nitrogen atoms and the carbon of a carbonyl group in addition to 4 other carbon ring atoms (CH₂ groups) from the original piperidine ring. In general, the ring system may be saturated or may be partially unsaturated, i.e., the ring system may contain one or more non-aromatic C—C or C—N double bonds.

The term “optionally substituted” means that group in question may be unsubstituted or it may be substituted one or several times, such as 1 to 3 times or 1 to 5 times. For example, an alkyl group that is “optionally substituted” with 1 to 5 chloro atoms, may be unsubstituted, or it may contain 1, 2, 3, 4, or 5 chlorine atoms.

Typically, substituted chemical moieties include one or more substituents that replace hydrogen. Exemplary substituents include, for example, halo (e.g., F, Cl, Br, I), alkyl, cycloalkyl, alkylcycloalkyl, alkenyl, alkynyl, haloalkyl including trifluoroalkyl, aralkyl, aryl, heteroaryl, heteroarylalkyl, spiroalkyl, heterocyclyl, heterocycloalkyl, hydroxyl (—OH), alkoxyl, aryloxyl, aralkoxyl, nitro (—NO₂), cyano (—CN), amino (—NH₂), N-substituted amino (—NHR″), N,N-disubstituted amino (—N(R″)R″), carboxyl (—COOH), —C(═O)R″, —OR″, —C(═O)OR″, —C(═O)NHSO₂R″, —NHC(═O)R″, aminocarbonyl (—C(═O)NH₂), N-substituted aminocarbonyl (—C(═O)NHR″), N,N-disubstituted aminocarbonyl (—C(═O)N(R″)R″), thiolato (SR″), sulfonic acid and its esters (—SO₃R″), phosphonic acid and its mono-ester (—P(═O)(OR″)(OH) and di-esters (—P(═O)(OR″)(OR″), —S(═O)₂R″, —S(═O)₂NH₂, —S(═O)₂NHR″, —S(═O)₂NR″R″, —SO₂NHC(═O)R″, —NHS(═O)₂R″, —NR″S(═O)₂R″, —CF₃, —CF₂CF₃, —NHC(═O)NHR″, —NHC(═O)NR″R″, —NR″C(═O)NHR″, —NR″C(═O)NR″R″, —NR″C(═O)R″ and the like. In relation to the aforementioned substituents, each moiety “R” can be, independently, any of H, alkyl, cycloalkyl, alkenyl, aryl, aralkyl, heteroaryl, or heterocycloalkyl, or when (R″(R″)) is attached to a nitrogen atom, R″ and R″ can be taken together with the nitrogen atom to which they are attached to form a 4- to 8-membered nitrogen heterocycle, wherein the heterocycloalkyl ring is optionally interrupted by one or more additional —O—, —S—, —SO, —SO₂—, —NH—, —N(alkyl)-, or —N(aryl)-groups, for example. In certain embodiments, chemical moieties are substituted by at least one optional substituent, such as those provided hereinabove. In the present invention, when chemical moieties are substituted with optional substituents, the optional substituents are not further substituted unless otherwise stated. For example; when R¹ is an alkyl moiety, it is optionally substituted, based on the definition of “alkyl” as set forth herein. In some embodiments, when R^{I} is alkyl substituted with optional aryl, the optional aryl substituent is not further substituted.

When any variable occurs more than one time in any constituent or formula for a compound, its definition at each occurrence is independent of its definition at every other occurrence. Thus, for example, if the R⁵ group is shown to be substituted with 0-2 substituents, then said group may optionally be substituted with up to two substituents and each substituents is selected independently from the definition of optionally substituted defined above. Also, combinations of substituents and/or variables are permissible only if such combinations result in stable compounds.

When a bond to a substituent is shown to cross a bond connecting two atoms in a ring, then such substituent may be bonded to any atom on the ring having an attached hydrogen atom. When a substituent is listed without indicating the atom via which such substituent is bonded to the rest of the compound of a given formula, then such substituent may be bonded via any atom in such substituent. Combinations of substituents and/or variables are permissible only if such combinations result in stable compounds.

The term “chiral” refers to molecules which have the property of non-superimposability of the mirror image partner, while the term “achiral” refers to molecules which are superimposable on their mirror image partner.

The term “diastereomers” refers to stereoisomers with two or more centers of dissymmetry and whose molecules are not mirror images of one another. The term “enantiomers” refers to two stereoisomers of a compound which are non-superimposable mirror images of one another. An equimolar mixture of two enantiomers is called a “racemic mixture” or a “racemate.” The term “isomers” or “stereoisomers” refers to compounds which have identical chemical constitution, but differ with regard to the arrangement of the atoms or groups in space.

Furthermore the indication of configuration across a carbon-carbon double bond can be “Z” referring to what is often referred to as a “cis” (same side) conformation whereas “E” refers to what is often referred to as a “trans” (opposite side) conformation. Regardless, both configurations, cis/trans and/or Z/E are contemplated for the compounds for use in the present invention. With respect to the nomenclature of a chiral center, the terms “d” and “I”, “R” and “S”, configuration are as defined by the IUPAC Recommendations. As to the use of the terms, diastereomer, racemate, epimer and enantiomer, these will be used in their normal context to describe the stereochemistry of preparations.

Unless specifically stated herein, the compounds as described herein may contain any stereoisomer, racemate, or a mixture thereof.

Natural amino acids represented by the compounds utilized in the present invention are in the “L” configuration, unless otherwise designated. Unnatural or synthetic amino acids represented by the compounds utilized in the present invention may be in either the “D” or “L” configurations.

Another aspect is a radiolabeled compound of any of the formulae delineated herein. Such compounds have one or more radioactive atoms (e.g., ³H, ²H, ¹⁴C, ¹³C, ³⁵S, ³²P, ¹²⁵I, ¹³¹I) introduced into the compound. Such compounds are useful for drug metabolism studies and diagnostics, as well as therapeutic applications.

“Substituted” refers to a group in which one or more hydrogen atoms are each independently replaced with the same or different substituent(s).

Terminology related to “protecting”, “deprotecting” and “protected” functionalities occurs throughout this application. Such terminology is well understood by persons of skill in the art and is used in the context of processes, which involve sequential treatment with a series of reagents. In that context, a protecting group refers to a group that is used to mask a functionality during a process step in which it would otherwise react, but in which reaction is undesirable. The protecting group prevents reaction at that step, but may be subsequently removed to expose the original functionality. The removal or “deprotection” occurs after the completion of the reaction or reactions in which the functionality would interfere. Protection and deprotection of functional groups may be performed by methods known in the art (see, for example, Green and Wuts Protective Groups in Organic Synthesis. John Wiley and Sons, New York, 1999.). In addition to the protecting groups as described elsewhere, hydroxyl or amino groups may be protected with any hydroxyl or amino protecting group. The amino protecting groups may be removed by conventional techniques. For example, acyl groups, such as alkanoyl, alkoxycarbonyl and aroyl groups, may be removed by solvolysis, e.g., by hydrolysis under acidic or basic conditions. Arylmethoxycarbonyl groups (e.g., benzyloxycarbonyl) may be cleaved by hydrogenolysis in the presence of a catalyst such as palladium-on-charcoal.

### Fluorinated Arenes and Other Compounds

Regioselective fluorination of arenes is enormously important for many areas in synthetic chemistry, including drug discovery because aryl fluoride moieties often improves potency and drugability of drug candidate. However, practical methods for introducing fluorines into arenes are extremely lacking. Embodiments of the invention describe a novel method for regioselectivity replacing C—H bonds directly by C—F bonds.

In general, the method comprises reacting a compound having the formula (I) in the presence of a palladium(II) catalyst and a promoter having the formula (II), with a fluorinating agent as fluorine source to form at least one of the ortho-fluorinated compounds having the formulae (IV) and (V) as shown in the following reaction scheme:

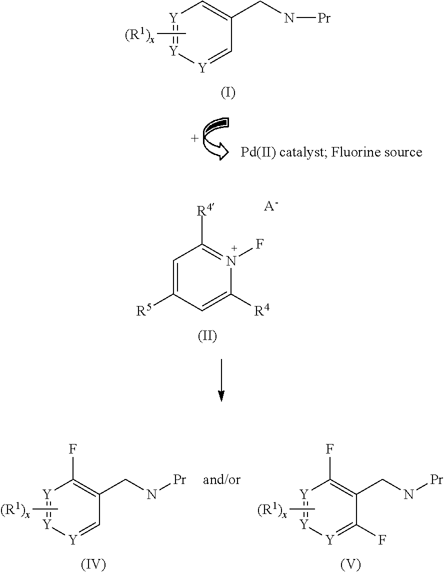

where the variables in formulae (I), (IV) and (V) have the following meanings:

each Y is C, CR¹, CH, CH₂, N, O, or S, wherein if Y is N, O, or S, at least one ring atom adjacent to Y is CR¹;

each R¹ is independently selected from the group of radicals consisting of C₁₋₂₀ alkyl, C₂₋₂₀ alkenyl, C₂₋₂₀ alkynyl, C₁-C₂₀ alkoxy, C₆-C₂₀ aryl, C₇-C₂₀ alkylaryl, C₄-C₂₀ heterocycle, C₄-C₂₀ heteroaryl, C₄-C₂₀ alkylheterocycle, C₇-C₂₀ alkylheteroaryl, C₆-C₂₀ aryloxy, —OH, —CO, —COOH, —CN, —N₃, halo, —CF₃, —OCF₃, —NH₂, —NO₂, —NR³R^{3′}, —N(O)R³, —SH, —SR³, —SOR³, —SO₂R³, —C(O)R³, —CO₂R³, —C(O)NR³R^{3′}, and —OC(O)R³; wherein the alkyl, alkenyl, alkynyl, alkoxy, aryl, heterocycle, heteroaryl, or aryloxy may be substituted or unsubstituted and wherein in the alkyl portion one or more —CH₂—, —CH₂CH₂—, or —(CH)ₙ groups, wherein n is equal to or greater than 1, and are each optionally replaced by —O— or —NH—,

or, wherein two R¹ are joined together to form a bicyclic or tricyclic alkyl or aryl with the ring to which they are attached, wherein if the bicyclic or tricyclic alkyl or aryl is a heterocycle, at least one ring atom adjacent to the heteroatom is substituted;

R³ and R^{3′} are each independently selected from the group of radicals consisting of H, C₁₋₂₀ alkyl, C₂₋₂₀ alkenyl, C₂₋₂₀ alkynyl, C₆-C₂₀ aryl, C₇-C₂₀ alkylaryl, C₄-C₂₀ heterocycle, C₄-C₂₀ heteroaryl, C₄-C₂₀ alkylheterocycle, and C₇-C₂₀ alkylheteroaryl; wherein the alkyl, alkenyl, alkynyl, aryl, heterocycle, or heteroaryl, be substituted or unsubstituted and wherein in the alkyl portion one or more, —CH₂—, —CH₂CH₂— groups are each optionally replaced by —O— or —NH—;

Pr is a protecting group; and

x is 0, 1, 2, 3, or 4.

In one embodiment, each Y is CH or CR¹. In another embodiment, one Y is —N— and the remaining Y groups are —CH₂CH₂—, CH₂, —(CH₂)ₙ— or CR¹. As understood herein, when Y is O or S, the heteroaryl of formula (I) will have single bonds adjacent to the O or S. n is an integer greater than or equal to 1.

In another embodiment, each Y is CH, CR¹, or an aromatic ring.

When the compounds of formulae (I), (IV) and (V) contain a heteroatom (i.e., the six-membered aromatic ring represents a pyridyl ring), the heteroatom must be shielded. A shielded heteroatom has one or both ring atoms adjacent to the heteroatom substituted with a group that can shield the heteroatom from the fluorination reaction, thus, at least one ring atom adjacent to this Y group is CR¹. In one embodiment, the shielding R¹ group is C₁₋₆ alkyl or a halogen.

In some embodiments, R¹ is preferably Cl or Br. This is particularly useful for further synthetic elaborations.

In another embodiment, each R¹ is independently a heteroaryl optionally substituted with one or more of alkyl, cycloalkyl, hydroxyl, halo, haloalkyl, amino, cyano, alkoxy, aroylalkyl, arylamino; anaryl optionally substituted with one or more of halo, hydroxyl, alkoxy, alkyl, haloalkyl, haloalkoxy, amino, cyano, aryl, heteroaryl, carboxy, amido, aryloxy, or methylenedioxy; an aryloxy, hydroxyalkyl, dihydroxyalkyl, alkylcarbonyl, cycloalkylcarbonyl, alkoxycarbonyl, amido, alkylamido, aminoalkylamido, alkylaminoalkylamido, dialkylaminoalkylamido, aminoalkyl, alkylaminoalkyl, dialkylaminoalkyl, amidoalkyl, alkylamidoalkyl, arylamidoalkyl, aralkylaminoalkyl, carboxy, alkoxycarbonyl, carboxyalkyl, amino, alkylamino, dialkylamino, aminoalkylamino, alkylaminoalkylamino, dialkylaminoalkylamino; a heterocyclylalkylamino optionally substituted with hydroxyl, alkoxy, alkyl, amino, alkylamino, dialkylamino, heterocyclyl, haloalkyl, aryl, heteroaryl; a heteroarylamino optionally substituted with alkyl, halo, hydroxyl, alkoxy, alkyl, haloalkyl, haloalkoxy, amino, cyano, aryl, heteroaryl, carboxy, aryloxy, amido; an arylamino optionally substituted with halo, hydroxyl, alkoxy, alkyl, haloalkyl, haloalkoxy, amino, cyano, aryl, heteroaryl, carboxy, amido, aryloxy; an alkanoyl; arylcarbonyl; aralkylcarbonyl; a heterocyclyl optionally substituted with alkyl, hydroxyl, amino, halo, alkoxy, haloalkyl, haloalkoxy, cyano, aryl, heteroaryl, carboxy, amido, aryloxy; a heterocyclylakyl optionally substituted with alkyl, hydroxyl, amino, halo, alkoxy, haloalkyl, haloalkoxy, cyano, aryl, heteroaryl, carboxy, amido, aryloxy, heteroarylalkyl, heterocyclylalkylaminoalkyl; a heterocyclylalkylamido optionally substituted with hydroxyl; heterocyclylcarbonylalkyl; a heterocyclylcarbonyl optionally substituted with alkyl, hydroxyl, amino, cyano, alkoxy; or an alkoxycarbonylalkyl, arylamido optionally substituted with halo, alkyl, alkoxy, amino, cyano, dialkylamino, heterocyclyl.

In one embodiment, x is 0, 1, or 2. In another embodiment, each R¹ is independently selected from the group of radicals consisting of C₁₋₆ alkyl, C₂₋₆ alkenyl, C₂₋₆ alkynyl, C₁-C₆ alkoxy , —OH, —CO, —COOH, —CN, —N₃, halo, —CF₃, —OCF₃, —NH₂, —NO₂, —NR³R^{3′}, —N(O)R³, —SH, —SR³, —SOR³, —SO₂R³, —C(O)R³, —CO₂R³, —C(O)NR³R^{3′}, and —OC(O)R³; wherein the alkyl, alkenyl, alkynyl, or alkoxy, may be substituted or unsubstituted and wherein in the alkyl portion one or more: —CH₂—, —CH₂CH₂— groups are each optionally replaced by —O— or —NH—; and R³ and R^{3′} are each independently selected from the group of radicals consisting of H, C₁₋₆ alkyl, C₂₋₆ alkenyl, and C₂₋₆ alkynyl.

In yet another embodiment, the compound of formula (I) is modified such that the methylene group alpha to the ring is substituted. For example, the substitution may be a C₁₋₆ alkyl.

Pr may be any amine protecting group that provides a sufficient yield in the reaction. In a preferred embodiment, the amine protecting group may be a sulfonyl moiety such as triflate (trifluoro-methanesulfonyl; Tf), sulfonyl trifluoroacetyl (TFA), 4-methoxy-2,3,6-trimethylbenzene sulphonyl (Mtr), methane sulfonyl, or toluene sulfonyl; or other protecting groups such as carbobenzoxy (CBZ), t-butoxycarbonyl (BOC), and 9-fluorenylmethoxycarbonyl (Fmoc). Other suitable amine protecting groups are given in Greene, “Protecting Groups in Organic Synthesis,” John Wiley and Sons, Second Edition (1991).

In some embodiments, the protecting group is preferably Tf, SA, or TFA. In another embodiment, the protecting group is Tf.

The present invention involves palladium-catalyzed reaction. In principle, any known palladium(H) catalyst may be used. In one embodiment, the catalyst is 5% or more palladium(II). However, other catalysts having a faster reaction rate are preferred. For example, in some preferred embodiments, catalyst is Pd(OAc)₂, Pd(CH₃CN)₄(OTs)₂, Pd(CH₃CN)₄(OTf)₂, Pd(NTf₂)₂, Pd(OTf)₂, or a combination of two or more thereof. In yet another embodiment, the catalyst is Pd(NTf₂)₂ and/or Pd(OTf)₂.

The catalyst is generally employed in catalytically effective amounts. See, for example the Examples section which follows.

Embodiments of the invention describe a novel method for regioselectivity replacing C—H bonds directly by fluorines.

The directing group used to control the regioselectivity can be subsequently converted to a wide range of synthetically desirable functional groups, thereby make this method extremely versatile for make a variety of fluorinated molecules of pharmaceutical interests.

The scheme below is illustrative of an embodiment of a method for expedient ortho-fluorination of triflamide-protected benzylamines:

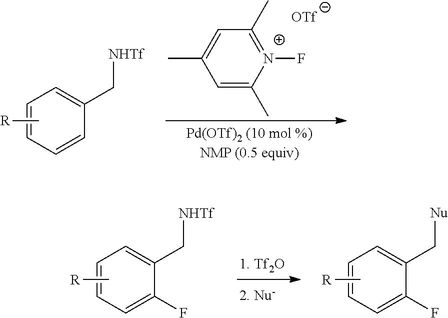

As discussed above, embodiments of the present invention involve palladium-catalyzed reactions. Any known palladium (II) catalyst may be used. In one embodiment, the catalyst is 5% or more palladium (II). However, other catalysts having a faster reaction rate are preferred. For example, in some preferred embodiments, catalyst is Pd(OAc)₂, Pd(CH₃CN)₄(OTs)₂, Pd(CH₃CN)₄(OTf)₂, Pd(NTf₂)₂, Pd(OTf)₂, or a combination thereof. In yet another embodiment, the catalyst is Pd(NTf₂)₂ or Pd(OTf)₂.

In a preferred embodiment, the palladium-catalyzed reaction provides ortho-fluorination for compounds of formula (I)

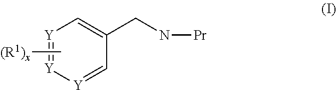

wherein:
each Y is CR¹, CH, N, O, or S, wherein if Y is N, O, or S, at least one ring atom adjacent to Y is CR¹;
each R¹ is independently selected from the group of radicals consisting of C₁₋₂₀ alkyl, C₁₋₂₀ alkyenyl, C₁₋₂₀ alkynyl, C₁-C₂₀ alkoxy, C₆-C₂₀ aryl, C₇-C₂₀ alkylaryl, C₄-C₂₀ heterocycle, C₄-C₂₀ heteroaryl, C₄-C₂₀ alkylheterocycle, C₇-C₂₀ alkylheteroaryl, C₆-C₂₀ aryloxy, —OH, —CO, —COOH, —CN, —N₃, halo, —CF₃, —OCF₃, —NH₂, —NO₂, —NR³R^{3′}, —N(O)R³, —SH, —SR³, —SOR³, —SO₂R³, —C(O)R³, —CO₂R³, —C(O)NR³R^{3′}, and —O₂CR³; wherein the alkyl, alkenyl, alkynyl, alkoxy, aryl, heterocycle, heteroaryl, or aryloxy may be substituted or unsubstituted and wherein in the alkyl portion one or more —CH₂—, —CH₂CH₂—, —(CH₂)n- groups are each optionally replaced by —O— or —NH—, wherein n is an integer equal to or greater than 1;
or wherein two R¹ are joined together to form a bicyclic or tricyclic alkyl or aryl with the ring to which they are attached, wherein if the bicyclic or tricyclic alkyl or aryl is a heterocycle, at least one ring atom adjacent to the heteroatom is substituted;
R³ and R^{3′} are each independently selected from the group of radicals consisting of H, C₁₋₂₀ alkyl, C₁₋₂₀ alkyenyl, C₁₋₂₀ alkynyl, C₆-C₂₀ aryl, C₇-C₂₀ alkylaryl, C₄-C₂₀ heterocycle, C₄-C₂₀ heteroaryl, C₄-C₂₀ alkylheterocycle, and C₇-C₂₀ alkylheteroaryl; wherein the alkyl, alkenyl, alkynyl, aryl, heterocycle, or heteroaryl, be substituted or unsubstituted and wherein in the alkyl portion one or more —CH₂CH₂— groups are each optionally replaced by —O— or —NH—;
Pr is a protecting group; and
x is 0, 1, 2, 3, or 4.

In one embodiment, each Y is CH or CR¹. In another embodiment, one Y is —N— and the remaining Y groups are CH or CR¹. As understood herein, when Y is O or S, the heteroaryl of formula (I) will have single bonds adjacent to the O or S.

When the compound of formula (I) contains a heteroatom (i.e., it is a pyridyl ring), the heteroatom must be shielded. A shielded heteroatom has one or both ring atoms adjacent to the heteroatom substituted with a group that can shield the heteroatom from the fluorination reaction, thus, at least one ring atom adjacent to this Y group is CR¹. In one embodiment, the shielding R¹ group is C₁₋₆ alkyl or a halogen.

Pr may be any amine protecting group that provides a sufficient yield in the reaction. In a preferred embodiment, the amine protecting group may be a sulfonyl moiety such as triflate (trifluoro-methanesulfonyl; TO, sulfonyl trifluoroacetyl (TFA), 4-methoxy-2,3,6-trimethylbenzene sulphonyl (Mtr), methane sulfonyl, or toluene sulfonyl; or other protecting groups such as carbobenzoxy (CBZ), t-butoxycarbonyl (BOC), and 9-fluorenylmethoxycarbonyl (Fmoc). Other suitable amine protecting groups are given in Greene, “Protecting Groups in Organic Synthesis,” John Wiley and Sons, Second Edition (1991).

In some embodiments, the protecting group is preferably Tf, SA, or TFA. In another embodiment, the protecting group is Tf.

In one embodiment, x is 0, 1, or 2. In another embodiment, each R¹ is independently selected from the group of radicals consisting of C₁₋₆ alkyl, C₁₋₆ alkyenyl, C₁₋₆ alkynyl, C₁-C₆ alkoxy , —OH, —CO, —COOH, —CN, —N₃, halo, —CF₃, —OCF₃, —NH₂, —NO₂, —NR³R^{3′}, —N(O)R³, —SH, —SR³, —SOR³, —SO₂R³, —C(O)R³, —CO₂R³, —C(O)NR³R^{3′}, and —O₂CR³; wherein the alkyl, alkenyl, alkynyl, or alkoxy, may be substituted or unsubstituted and wherein in the alkyl portion one or more —CH₂CH₂— groups are each optionally replaced by —O— or —NH—; and R³ and R^{3′} are each independently selected from the group of radicals consisting of H, C₁₋₆, alkyl, C₁₋₆ alkyenyl, and C₁₋₆ alkynyl.

In yet another embodiment, the compound of formula (I) is modified such that the methylene group alpha to the ring is substituted. For example, the substitution may be a C₁₋₆ alkyl.

In one embodiment, any fluorinating reagent may be used. In one embodiment, the fluorinating agent is an electrophilic fluorinating agent.

In another embodiment, the fluorinating reagent for use in the fluorination reaction has the formula:

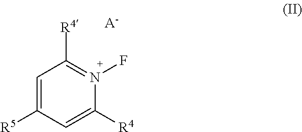

wherein
A⁻ is a counter ion; and
R⁴, R^{4′}, and R⁵ are each independently halogen, C₁₋₁₂ alkyl, or C₂₋₁₂ alkenyl, wherein the alkyl or alkenyl may be substituted with one or more halogen.

In one embodiment, wherein the fluorinating reagent has formula (II), A⁻ is OTf⁻, BF₄⁻, or PF₆⁻ and R⁴ and R^{4′} are each independently C₁₋₆ alkyl, and R⁵ is H or a C₁₋₆alkyl.

In yet another embodiment, the fluorinating reagent for use in the fluorination reaction has the formula:

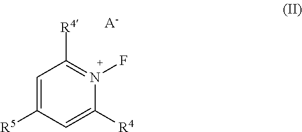

wherein
A⁻ is OTf⁻, BF₄⁻, or PF₆⁻;
R⁴ and R^{4′} are each independently a C₁₋₆ alkyl, and
R⁵ is H or a C₁₋₆ alkyl.

In one embodiment, A is OTf⁻. In another embodiment, the fluorinating reagent is N-fluoro-2-4-6-trimethylpyridinium triflate. In one embodiment, 1-5 equivalents of the fluorinating reagent is used.

A promoter is also used in the fluorination reaction, where the promoter has the formula

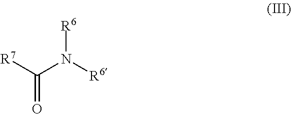

wherein
R⁶ and R^{6′} are each H or a C₁₋₆ alkyl, wherein at least one of R⁶ and R^{6′} is not H,
R⁷ is H, or a C₁₋₆ alkyl,
or wherein either R⁶ and R^{6′} together with the nitrogen to which they are attached or R⁶ and R⁷ together with the nitrogen and carbonyl to which they are attached form a 5- or 6-membered ring which may be unsubstituted or substituted with one or more C₁₋₆ alkyl groups;

In one embodiment, the promoter is N-methylpyrrolidinone (NMP). It has been found that this compound is particularly useful to increase both the rate of the reaction and the yield for a number of arenes.

In another embodiment, the promoter is N-methylpyrrolidinone (NMP) and the catalyst is Pd(OTf)₂.

In yet another embodiment, the promoter is DMF. The best yield of mono-fluorinated product as defined below in the Examples, Table 1 as compound 2a was 41% (˜7% 2aa) when the reaction was performed when the promoter was 0.5 equiv. of DMF.

In one embodiment, 0.1-5 equivalents of the promoter is used. In another embodiment, 0.3-0.7 equivalents of the promoter is used. In one embodiment, the identity and amount of promoter is adjusted to obtain the optimal yield of the preferred product.

In general, any fluorinating reagent may be used. In one embodiment, the fluorinating agent is an electrophilic fluorinating agent.

In another embodiment, the fluorinating reagent for use in the fluorination reaction has the formula:

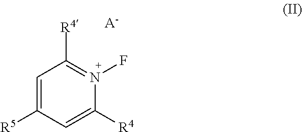

wherein
A⁻ is a counter ion; and
R⁴, R^{4′}, and R⁵ are each independently halogen, C₁₋₁₂ alkyl, or C₂₋₁₂ alkenyl, wherein the alkyl or alkenyl may be substituted with one or more halogen.

In one embodiment, wherein the fluorinating reagent has formula (II), A⁻ is OTf⁻, BF₄⁻, or PF₆⁻ and R⁴ and R^{4′} are each independently C₁₋₆ alkyl, and R⁵ is H or a C₁₋₆ alkyl.

In yet another embodiment, the fluorinating reagent for use in the fluorination reaction has the formula:

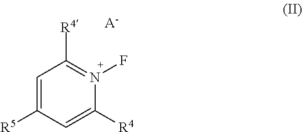

wherein
A⁻ is OTf⁻, BP₄⁻, or PF₆⁻;
R⁴ and R^{4′} are each independently a C₁₋₆ alkyl, and
R⁵ is H or a C₁₋₆ alkyl.

In one embodiment, A is OTf⁻. In another embodiment, the fluorinating reagent is N-fluoro-2-4-6-trimethylpyridinium triflate. In one embodiment, 1-5 equivalents of the fluorinating reagent are used.

In some preferred embodiments, the fluorine source comprises N-fluoro-2,4,6-trimethylpyridinium triflate and the promoter comprises NMP.

In other preferred embodiments, the catalyst is Pd(OTf)₂, the compound of formula (I) comprises triflamide as a convertible directing group, and N-methylpyrrolidinone (NMP) is employed as the ligand to promote the reaction.

It is believed that oxidation of L₂PdArI (L generically represents a lignad) by a fluorine source via a S_{N}2-type mechanism gives a cationic pentacoordinated L₂Pd(IV)ArIF complex (Furuya, T.; Ritter, T. J. Am. Chem. Soc. 2008, 130, 10060). Without wishing to be bound by theory, an analogous Pd(IV) intermediate could be involved in the fluorination reactions. The combination of the triflamide and catalytic amount of a promoter such as NMP is important for the formation of such an intermediate.

The reaction is generally carried out in an aprotic solvent, which may be apolar or polar. Protic solvents solvate anions (negatively charged solutes) strongly via hydrogen bonding. Water is a protic solvent. Aprotic solvents such as acetone or dichloromethane tend to have large dipole moments (separation of partial positive and partial negative charges within the same molecule) and solvate positively charged species via their negative dipole. In chemical reactions the use of polar protic solvents favors the SN₁ reaction mechanism, while polar aprotic solvents favor the SN₂ reaction mechanism. Any aprotic solvent can be used.

In one embodiment, the solvent is 1,1-dichloroethene (DCE) or PhCF₃.

In another preferred embodiment, any of the reactions described herein can be carried out in a wide range of temperatures. For example, a reaction may be carried out at any temperature of from room temperature (about 23° C.) to 180° C., or up to the boiling temperature of the solvent.

In one embodiment, the catalytic turnover and the reaction time takes between about 5 minutes to 12 hours. In another embodiment, the reaction takes 5 minutes to 4 hours. In yet another embodiment, the reaction takes no more than 60 minutes, and in yet another embodiment, the reaction takes no more than 20 minutes.

In general, the amount of monofluoronated and difluoronated product can be optimized through selection of the starting material and/or catalyst. For example, in some embodiments, the use of the catalyst Pd(NTf₂)₂ or Pd(OTf)₂ gives mainly difluorinated product. Fluorination of meta-substituted arenes gives mono-fluorinated products predominantly.

In further embodiments, the yield of difluorinated product is at least 30%. In yet another embodiment, the yield of difluorinated product is at least 50%. In another embodiment, the yield of monofluorinated product is at least 30%. In another embodiment, the yield of monofluorinated product is at least 40%. In another embodiment, the yield of monofluorinated product is at least 60%.

In general, the N-protected amino group represented as N-Pr in formula (I) controls the regioselectivity of the fluorination and, thus, serves as a directing group. The directing group of formulae (IV) and (V) can be converted to a wide range of synthetically desirable functional groups, which makes this method extremely versatile for preparing a variety of fluorinated molecules of pharmaceutical interest. The conversion of triflamide into a wide variety of synthetically useful functional groups makes this fluorination protocol broadly applicable in medicinal chemistry and synthesis.

The scheme below is illustrative of an embodiment of a method for expedient ortho-fluorination of triflamide-protected benzylamines and subsequent conversion of the directing group:

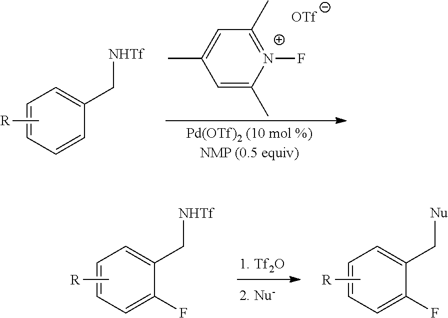

A further advantage of the methods as provided herein is that it is not necessary to use microwave heating in the reaction.

[00901 Other advantages of the methods described herein, include direct fluorination of C—H bonds, rather than going through bromination then displacement; the directing group can be converted to a broad range of functional groups such as aldehydes, azides, nitriles and alkyl carboxylic acids, allowing the synthesis of a variety of fluorine-containing compounds, for example, pyridines; the reaction rate is extremely fast; and can be expanded to achieve large scale production. The fluorine-containing compounds are particularly useful as drug candidates, either as part of a screening assay or as a therapeutic agent.

The catalyzed reaction as described herein, when using a fluorine-18 source, allows for the facile incorporation of ¹⁸F into the benzylamine. This is useful, for example, for positron emission tomography which is an extremely important technology for noninvasive molecular imaging, particularly for potential drug products. Incorporation of ¹⁸F into the benzylamine is particularly useful when the fluorination reaction occurs quickly, i.e, less than one hour and, in one embodiment, less than 20 minutes.

Reaction Products 100921 The fluorination reaction has wide ranging utility allowing for the synthesis of a variety of fluorinated arenes, especially drug-like heterocyles. For example, elagolix, a current Phase II clinical compound for treatment of endometriosis contains ortho-fluorinated benzylamines. The screening used to obtain this compound was largely limited due to the lack of fluorinated benzylamines, however, the methods described herein, can readily provide a broad range of these compounds and improve the biological properties for this and other screens wherein fluorinated benzylamines have activity for the targeted biologic agent.

Another embodiment of the present invention comprises a method of making a fluorinated compound comprising:

reacting a compound having the formula (I);

a palladium(II) catalyst;

a fluorinating reagent having the formula (II); and

a promoter having the formula (III),
to form at least one of the ortho-fluorinated compounds having the formulas (IV) and (V), wherein each of formulas (I), (II), (III), (IV) and (V) are described hereinabove, and
reacting the ortho-fluorinated compound of formula (IV) or (V) with a reagent to obtain at least one of the compounds having the formula:

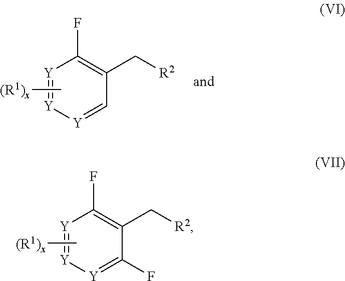

wherein
each R² is independently selected from the group of radicals consisting of C₁₋₂₀ alkyl, C₂₋₂₀ alkenyl, C₂₋₂₀ alkynyl, C₁-C₂₀ alkoxy, C₆-C₂₀ aryl, C₇-C₂₀ alkylaryl, C₄-C₂₀ heterocycle, C₄-C₂₀ heteroaryl, C₄-C₂₀ alkylheterocycle, C₇-C₂₀ allcylheteroaryl, C₆-C₂₀ aryloxy, —OH, —CO, —COOH, —CN, —N₃, halo, —CF₃, —OCF₃, —NH₂, —NO₂, —NR³R^{3′}, —N(O)R³, —SH, —SR³, —SOR³, —SO₂R³, —C(O)R³, —CO₂R³, —C(O)NR³R^{3′}, and —OC(O)R³; wherein the alkyl, alkenyl, alkynyl, alkoxy, aryl, heterocycle, heteroaryl, or aryloxy be substituted or unsubstituted and wherein in the alkyl portion one or more: —CH₂—, —CH₂CH₂—, —(CH₂)ₙ— groups are each optionally replaced by —O— or —NH—; wherein n is an integer equal to or greater than 1; and
R³ and R^{3′} are each independently selected from the group of radicals consisting of H, C₁₋₂₀ alkyl, C₂₋₂₀ alkenyl, C₂₋₂₀ alkynyl, C₆-C₂₀ aryl; C₇-C₂₀ alkylaryl, C₄-C₂₀ heterocycle, C₄-C₂₀ heteroaryl, C₄-C₂₀ allcylheterocycle, and C₇-C₂₀ alkylheteroaryl; wherein the alkyl, alkenyl, alkynyl, aryl, heterocycle, or heteroaryl, be substituted or unsubstituted and wherein in the alkyl portion one or more :—CH₂—, —CH₂CH₂—, groups are each optionally replaced by —O— or —NH—; wherein n is an integer equal to or greater than 1,
   wherein R¹, Y and x are the same as discussed above.

In embodiment, the compound of formula (IV) or (V) is isolated before reacting to form the compound of formula (VI) or (VII). In another embodiment, the product is substantially the compound of formula (VI). In another embodiment, the product is substantially the compound of formula (VII).

The ortho-fluorinated compounds of formula (VI) or (VII) can be readily converted, e.g., as shown in the reaction scheme below:

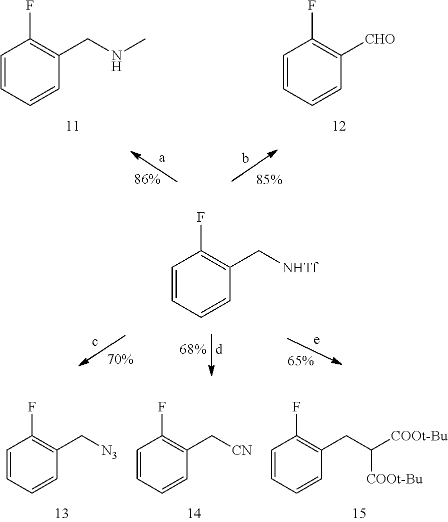

The triflamide group can be readily removed by any method known in the art. For example, the triflamide may be removed by Hendrickson's procedure (i.e, reacting with one equivalent of LiA1H₄ in diethyl ether under reflux. (J. B. Hendrickson, R. Bergeron, Tetrahedron Lett. 1973, 14, 3839).

To minimize the restriction that the fluorine is introduced onto ortho positions to a particular directing group, triflamides are converted to a broad range of synthetically useful functional groups exploiting known reactivities. These transformations allow access to at least five major classes of ortho-fluorinated synthons, namely, benzaldehyde, benzylamine, benzylazide, phenylacetonitrile and phenylpropanoate, thus greatly expanding the scope of ArF synthesis. The ortho-fluorinated phenylpropionic acids are especially valuable as they can not be accessed by either ortho-lithiation or by palladation using previously reported directing groups.

In another preferred embodiment, a compound obtained by embodiments of the methods described herein is a therapeutic agentg selected from: an anti-proliferative agent, an anti-inflammatory agent, an immunomodulatory agent, a neurotrophic factor, an agent for treating cardiovascular disease, an agent for treating liver disease, an anti-viral agent, an agent for treating blood disorders, an agent for treating diabetes, and an agent for treating immunodeficiency disorders.

In one embodiment, the compOunds made by the methods as described herein encompass various isomeric forms. Such isomers include, e.g., stereoisomers, e.g., chiral compounds, e.g., diastereomers and enantiomers, e.g. racemates. “Racemate” is an equimolar mixture of a pair of enantiomers. A racemate does not exhibit optical activity. The chemical name or formula of a racemate is distinguished from those of the enantiomers by the prefix (±)- or rac- (or racem-) or by the symbols RS and SR.

The present invention further encompasses salts, solvates, prodrugs and active metabolites.

The term “salts” can include acid addition salts or addition salts of free bases. Preferably, the salts are pharmaceutically acceptable. Examples of acids which may be employed to form pharmaceutically acceptable acid addition salts include, but are not limited to, salts derived from nontoxic inorganic acids such as nitric, phosphoric, sulfuric, or hydrobromic, hydroiodic, hydrofluoric, phosphorous, as well as salts derived from nontoxic organic acids such as aliphatic mono- and dicarboxylic acids, phenyl-substituted alkanoic acids, hydroxyl alkanoic acids, alkanedioic acids, aromatic acids, aliphatic and aromatic sulfonic acids, and acetic, maleic, succinic, or citric acids. Non-limiting examples of such salts include napadisylate, besylate, sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate, galacturonate (see, for example, Berge, et al. “Pharmaceutical Salts,” J. Pharma. Sci. 1977;66:1).

The phrase “pharmaceutically acceptable,” as used in connection with those compounds, materials, compositions, and/or dosage forms that are within the scope of sound medical judgment, suitable for contact with the tissues of human beings and animals without excessive toxicity, irritation, allergic response, or other problems or complications commensurate with a reasonable benefit/risk ratio. Preferably, as used herein, the term “pharmaceutically acceptable” means approved by a regulatory agency of the federal or a state government or listed in the U.S. Pharmacopoeia or other generally recognized pharmacopeias for use in mammals, and more particularly in humans.

Typically, a pharmaceutically acceptable salt of a compound such as one made by the method of the present invention may be prepared by using a desired acid or base as appropriate. The salt may precipitate from solution and be collected by filtration or may be recovered by evaporation of the solvent. For example, an aqueous solution of an acid such as hydrochloric acid may be added to an aqueous suspension of a compound of formula I and the resulting mixture evaporated to dryness (lyophilized) to obtain the acid addition salt as a solid. Alternatively, a compound may be dissolved in a suitable solvent, for example an alcohol such as isopropanol, and the acid may be added in the same solvent or another suitable solvent. The resulting acid addition salt may then be precipitated directly, or by addition of a less polar solvent such as diisopropyl ether or hexane, and isolated by filtration.

The acid addition salts of the compounds may be prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine.

The base addition salts of said acidic compounds are prepared by contacting the free acid form with a sufficient amount of the desired base to produce the salt in the conventional manner. The free acid form may be regenerated by contacting the salt form with an acid and isolating the free acid.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compound of the invention are within the scope of the invention. The salts of the compound of formula I may form solvates (e.g., hydrates) and the invention also includes all such solvates. The meaning of the word “solvates” is well known to those skilled in the art as a compound formed by interaction of a solvent and a solute (i.e., solvation). Techniques for the preparation of solvates are well established in the art (see, for example, Brittain. Polymorphism in Pharmaceutical solids. Marcel Decker, New York, 1999.). Solvates may be represented, for example, by the formula R·(solvent), where R is a compound of the invention. A given compound may form more than one solvate including, for example, monosolvates (R(solvent)) or polysolvates (R(solvent)ₙ) wherein n is an integer) including, for example, disolvates (R(solvent)₂), trisolvates (R(solvent)₃), and the like, or hemisolvates, such as, for example, R(solvent)_{n/2}, R(solvent)_{n/3}, R(solvent)_{n/4} and the like wherein n is an integer. Solvents herein include mixed solvents, for example, methanol/water, and as such, the solvates may incorporate one or more solvents within the solvate.

The term “prodrug” includes compounds with moieties, which can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. Sci. 66:1-19; Silverman (2004) The Organic Chemistry of Drug Design and Drug Action, Second Ed., Elsevier Press, Chapter 8, pp. 497-549). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halogen, or methoxy substituents) aryl and aryl-lower alkyl esters, amides, lower-alkyl amides, di-lower alkyl amides, and hydroxy amides. Other prodrug moieties include propionoic and succinic acid esters, acyl esters and substituted carbamates. Prodrugs which are converted to active forms through other mechanisms in vivo are also included.

As used herein, the term “hydrate” refers to a compound of the present invention which is associated with water in the molecular form, i.e., in which the H—OH bond is not split, and may be represented, for example, by the formula R·H₂O, where R is a compound of the invention. A given compound may form more than one hydrate including, for example, monohydrates (R—H₂O) or polyhydrates (R(H₂O)ₙ) wherein n is an integer >1; including, for example, dihydrates (R(H₂O)₂), trihydrates (R(H₂O)₃), and the like, or hemihydrates, such as, for example, R(H₂O)_{n/2}, R(H₂O)_{n/3}, R(H₂O)_{n/4} and the like wherein n is an integer.

As used herein, the term “acid hydrate” refers to a complex that may be formed through association of a compound having one or more base moieties with at least one compound having one or more acid moieties or through association of a compound having one or more acid moieties with at least one compound having one or more base moieties, said complex being further associated with water molecules so as to form a hydrate, wherein said hydrate is as previously defined and R represents the complex herein described above.

A number of the compounds of the present invention and intermediates therefor exhibit tautomerism and therefore may exist in different tautomeric forms under certain conditions. As used herein, the term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. A specific example of a proton tautomer is an imidazole moiety where the hydrogen may migrate between the ring nitrogens. Valence tautomers include interconversions by reorganization of some of the bonding electrons. All such tautomeric forms (e.g., all keto-enol and imine-enamine forms) are within the scope of the invention. The depiction of any particular tautomeric form in any of the structural formulas herein is not intended to be limiting with respect to that form, but is meant to be representative of the entire tautomeric set.

Compounds described herein throughout, can be used or prepared in alternate forms. For example, many amino-containing compounds can be used or prepared as an acid addition salt. Often such salts improve isolation and handling properties of the compound. For example, depending on the reagents, reaction conditions and the like, compounds as described herein can be used or prepared, for example, as their hydrochloride or tosylate salts. Isomorphic crystalline forms, all chiral and racemic forms, N-oxide, hydrates, solvates, and acid salt hydrates, are also contemplated to be within the scope of the present invention.

Certain acidic or basic compounds of the present invention may exist as zwitterions. All forms of the compounds, including free acid, free base and zwitterions, are contemplated to be within the scope of the present invention. It is well known in the art that compounds containing both basic nitrogen atom and acidic groups often exist in equilibrium with their zwitterionic forms. Thus, any of the compounds described herein throughout that contain, for example, both basic nitrogen and acidic groups, also include reference to their corresponding zwitterions.

When the following abbreviations are used throughout this disclosure, they have the following meaning:

Ac acetyl

aq aqueous

BOC tert-butyloxycarbonyl

(Boc)₂O di-tert-butyldicarbonate

CBZ carbobenzoxy

CDCl₃ deuterated chloroform

DMF dimethyl formamide

ESI electrospray (mass spectrometry)

ESI-TOF electrospray/time of flight mass spectrometry

EtOAc ethyl acetate

Fmoc 9-fluorenylmethoxycarbonyl

HPLC high-performance liquid chromatography

Hz hertz

IR infra red spectroscopy

LC-MS liquid chromatography/mass spectroscopy

mg milligram(s)

MHz megahertz

min minute(s)

mL milliliter

mmol millimole(s)

mol mole

MS mass spectrometry

Ms methanesulfonyl

Mtr 4-methoxy-2,3,6-trimethylbenzene sulphonyl

NMP N-methylpyrrolidinone

NMR nuclear magnetic resonance

Ph phenyl

ppm parts per million

rt room temperature

Tf trifluoromethanesulfonyl

TFA trifluoroacetyl

THF tetrahydrofuran

TLC thin layer chromatography

TOF time of flight (mass spectrometry)

Ts toluensulfonyl

w/w weight per unit weight

A comprehensive list of abbreviations utilized by organic chemists (i.e. persons of ordinary skill in the art) appears in the first issue of each volume of the Journal of Organic Chemistry. The list, which is typically presented in a table entitled “Standard List of Abbreviations” is incorporated herein by reference.

Embodiments of the invention may be practiced without the theoretical aspects presented. Moreover, the theoretical aspects are presented with the understanding that Applicants do not seek to be bound by the theory presented.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments.

All documents mentioned herein are incorporated herein by reference. All publications and patent documents cited in this application are incorporated by reference for all purposes to the same extent as if each individual publication or patent document were so individually denoted. By their citation of various references in this document, applicants do not admit any particular reference is “prior art” to their invention.

Embodiments of inventive compositions and methods are illustrated in the following examples.

### EXAMPLES

The following non-limiting Examples serve to illustrate selected embodiments of the invention. It will be appreciated that variations in proportions and alternatives in elements of the components shown will be apparent to those skilled in the art and are within the scope of embodiments of the present invention.

General Information: Solvents were obtained from Acros or Aldrich and used directly without further purification. Infrared spectra were recorded on a Perkin Elmer FT-IR Spectrometer. NMR spectra were recorded on Varian-Inova (400 MHz for ¹H; 100 MHz for ¹³C; 376 MHz for ¹⁹F) and Bruker-DRX (500 MHz for ¹H; 125 MHz for ¹³C) instruments internally referenced to SiMea signal or chloroform. High resolution mass spectra were recorded at Center for Mass Spectrometry, The Scripps Research Institute. All the benzylamine were purchased from Oakwood, Sigma-Aldrich and Alfa-Aesar and used as received. All the fluorine reagents are commercial available from Alfa-Aesar, Sigma-Aldrich and TCI. N-methylpyrrolidinone (NMP) was obtained from Acros and used as received. Palladium acetate was purchased from Sigma-Aldrich. Pd(CH₃CN)₂(OTs)₂, Pd(CH₃CN)₄(OTf)₂, Pd(NTf₂)₂, and Pd(OTf)₂.2H₂O were synthesized following the reported methods.

TABLE 1
Optimization of Reaction Conditions

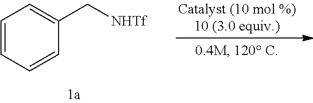

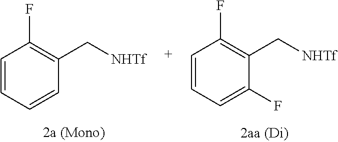

| | | | | | yield |
|---|---|---|---|---|---|
| | | Additive | | time | (%)^{a} |
| entry | Catalyst (mol%) | (equiv) | Solvent | (h) | (2a/2aa)^{b} |
| 1 | Pd(OAc)₂ | NMP (0.5) | DCE | 12 | 71 (1/2.5) |
| 2 | Pd(OAc)₂ | NMP (0.5) | t-BuOH | 12 | N.R. |
| 3 | Pd(OAc)₂ | NMP (0.5) | THF | 12 | 23 (1/1.6) |
| 4 | Pd(OAc)₂ | NMP (0.5) | EtOAc | 12 | 60 (1/1.3) |
| 5 | Pd(OAc)₂ | NMP (0.5) | PhCF₃ | 12 | 64 (1/4.3) |
| 6 | Pd(CH₃CN)₂(OTs)₂ | NMP (0.5) | DCE | 8 | 65 (1/1.2) |
| 7 | Pd(NTf₂)₂ | NMP (0.5) | DCE | 8 | 74 (1/4.9) |
| 8 | Pd(CH₃CN)₄(OTf)₂ | NMP (0.5) | DCE | 4 | 74 (1/4.5) |
| 9 | Pd(OTf)₂•2H₂O | NMP (0.5) | DCE | 4 | 80 (1/5.7) |
| 10 | Pd(OTf)₂•2H₂O | NMP (5.0) | DCE | 4 | 27 (1/3.7) |
| 11 | Pd(OTf)₂•2H₂O | NMP (1.0) | DCE | 4 | 76 (1/6.5) |
| 12 | Pd(OTf)₂•2H₂O | NMP (0.2) | DCE | 4 | 51 (1/0.7) |
| 13 | Pd(OTf)₂•2H₂O | NMP (0.1) | DCE | 4 | 37 (1/0.8) |
| 14 | Pd(OTf)₂•2H₂O | NMP (0.5) | PhCF₃ | 4 | 72 (1/12) |
| 15^{c} | Pd(OTf)₂•2H₂O | DMF (0.5) | DCE | 2 | 48 (5.6/1) |

^{a}Isolated yield.
^{b}the ratio of 2a/2aa was determined by crude ¹H NMR.
^{c}10 (2.0 equiv) was used.

### Example 1

### Synthesis of Trifluoromethanesulfonamides 1a-o and 2a

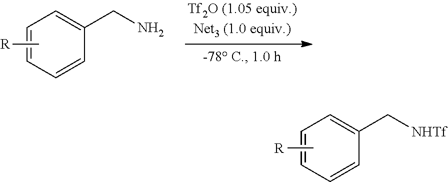

General Procedure: To a stirred solution of benzylamine (50 mmol, 1.0 equiv.) in dichloromethane (100 mL) was added triethylamine (7.0 mL, 50 mmol, 1.0 equiv.) at −78° C. under nitrogen. After stirring for 5 min at −78° C., trifluoromethanesulfonic anhydride (8.8 mL, 52.5 mmol, 1.05 equiv.) was added dropwise and the mixture was stirred for 1 h at that temperature before being quenched by water (100 mL). The organic layer was separated and the aqueous layer was extracted with dichloromethane (50 mL×2). The combined organic phase was washed with brine (100 mL), and then dried over Na₂SO₄. Evaporation and column chromatography on silica gel (ethyl acetate/hexane=1:100-1:5 as eluant) afforded corresponding trifluoromethanesulfonamides 1a-o and 2a as colorless or pale yellow oil or solid, giving a >90% yield in all cases.

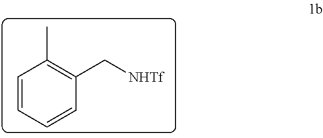

¹H NMR (400 MHz, CDCl₃) δ 7.30-7.21 (m, 4H), 4.82 (br, 1H), 4.46 (d, J=5.6 Hz, 2H), 2.38 (s, 3H); ¹³C NMR (100 MHz, CDCl₃) δ 136.5, 132.7, 131.0, 129.0, 128.9, 126.6, 119.7 (q, J_{C-F}=319.6 Hz), 46.3, 18.8; IR (neat) 3315, 2928, 1733, 1429, 1372, 1230, 1191, 1145, 1044, 878, 745, 597 cm⁻¹; HRMS (ESI-TOF) calcd. for C₉H₉F₃NO₂S ([M−H]⁻): 252.0312, Found: 252.0308.

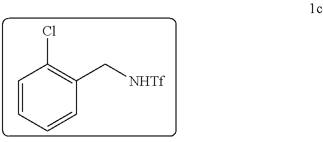

¹H-NMR (400 MHz, CDCl₃) δ 7.44-7.39 (m, 2H), 7.35-7.28 (m, 2H), 5.31 (br, 1H), 4.55 (d, J=6.0 Hz, 2H); ¹³C NMR (100 MHz, CDCl₃) δ 133.6, 133.0, 130.3, 130.2, 129.8, 127.5, 119.5 (q, J_{C-F}=319.3 Hz), 46.1; IR (neat) 3318, 2931, 1732, 1445, 1374, 1231, 1194, 1145, 1043, 1063, 850, 753, 612 cm⁻¹; HRMS (ESI-TOF) calcd. for

C₈H₆ClF₃NO₂S ([M−H]⁻): 271.9765, Found: 271.9760.

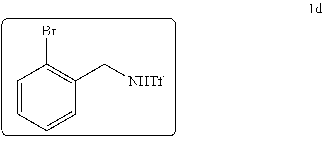

¹H NMR (400 MHz, CDCl₃) δ 7.60 (dd, J=8.0, 1.2 Hz, 1H), 7.42 (dd, J=7.6, 2.0 Hz, 1H), 7.35 (dt, J=7.6, 1.2 Hz, 1H), 7.24 (dt, J=7.6, 2.0 Hz, 1H), 5.35 (br, 1H), 4.54 (d, J=6.0 Hz, 2H); ¹³C NMR (100 MHz, CDCl₃) δ 134.7, 133.1, 130.5, 130.4, 128.2, 123.6, 119.5 (q, J_{C-F}=319.4 Hz), 48.4; IR (neat) 3320, 2360, 1736, 1440, 1374, 1230, 1193, 1144, 1056, 1029, 874, 751, 612 cm⁻¹; HRMS (ESI-TOF) calcd. for C₈H₆BrF₃NO₂S ([M−H]⁻): 315.9260, Found: 315.9264.

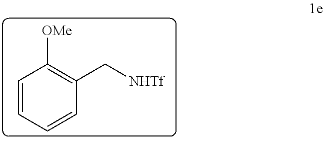

¹H NMR (400 MHz, CDCl₃) δ 7.34 (dt, J=8.0, 1.6 Hz, 1H), 7.23 (dd, J=7.2, 1.6 Hz, 1H), 6.96 (dt, J=7.6, 0.8 Hz, 1H), 6.91 (d, J=8.4 Hz, 1H), 5.49 (br, 1H), 4.42 (d, J=6.0 Hz, 2H), 3.89 (s, 3H); ¹³C NMR (100 MHz, CDCl₃) δ 157.4, 130.1, 129.7, 123.7, 120.8, 119.6 (q, J_{C-F}=319.4 Hz), 110.4, 55.3, 44.9; IR (neat) 3315, 2945, 2360, 1604, 1420, 1370, 1228, 1187, 1143, 1030, 871, 753, 616 cm⁻¹; HRMS (ESI-TOF) calcd. for C₉H₉F₃NO₃S ([M−H]⁻): 268.0261, Found: 268.0262.

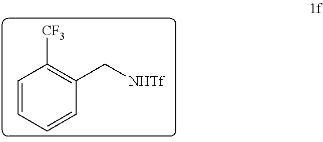

¹H NMR (400 MHz, CDCl₃) δ 7.71 (d, J=8.0 Hz, 1H), 7.65-7.61 (m, 2H), 7.53-7.48 (m, 1H), 5.11 (br, 1H), 4.62 (d, J=6.4 Hz, 2H); ¹³C NMR (100 MHz, CDCl₃) δ 133.5, 132.8, 131.0, 128.9, 128.4 (q, J_{C-F}=30.4 Hz), 126.4 (q, J_{C-F}=5.5 Hz), 121.4 (q, J_{C-F}=272.1 Hz), 119.6 (q, J_{C-F}=319.2 Hz), 44.8 (q, J_{C-F}=2.2 Hz); IR (neat) 3314, 2926, 2360, 1610, 1433, 1376, 1315, 1195, 1172, 1144, 1119, 1040, 854, 763, 603 cm⁻¹; HRMS (ESI-TOF) calcd. for C₉H₆F₆NO₂S ([M−H]⁻): 306.0029, Found: 306.0031.

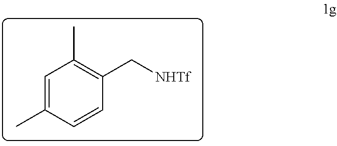

¹H NMR (400 MHz, CDCl₃) δ 7.15 (d, J=8.0 Hz, 1H), 7.04 (s, 1H), 7.03 (d, J=8.0 Hz, 1H), 4.72 (br, 1H), 4.41 (d, J=5.2 Hz, 2H), 2.34 (s, 3H), 2.32 (s, 3H); ¹³C NMR (100 MHz, CDCl₃) δ 138.9, 136.4, 131.8, 129.7, 129.0, 127.2, 119.7 (q, J_{C-F}=319.8 Hz), 46.1, 30.0, 18.7; IR (neat) 3311, 2925, 1617, 1426, 1371, 1231, 1200, 1145, 1041, 872, 610 cm⁻¹; HRMS (ESI-TOF) calcd. for C₁₀H₁₁F₃NO₂S ([M−H]⁻): 266.0468, Found: 266.0473.

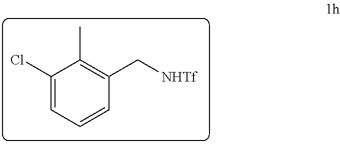

¹H NMR (400 MHz, CDCl₃) δ 7.39 (d, J=7.6 Hz, 1H), 7.22-7.17 (m, 2H), 4.81 (br, 1H), 4.49 (s, 2H), 2.42 (s, 3H); ¹³C NMR (100 MHz, CDCl₃) δ 135.8, 134.7, 134.6, 130.0, 127.4, 127.2, 119.6 (q, J_{C-F}=319.5 Hz), 46.8, 15.6; IR (neat) 3314, 2927, 2360, 1573, 1430, 1371, 1231, 1192, 1143, 1088, 1046, 857, 783, 606 cm^{-1;} HRMS (ESI-TOF) calcd. for C₉H₈ClF₃NO₂S ([M−H]⁻): 285.9922, Found: 285.9925.

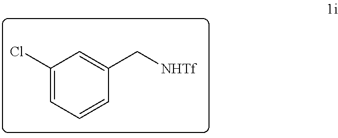

¹H NMR (400 MHz, CDCl₃) δ 7.33-7.30 (m, 3H), 7.23-7.20 (m, 1 H), 5.20 (br, 1H), 4.42 (d, J=6.0 Hz, 2H); ¹³C NMR (100 MHz, CDCl₃) δ 137.2, 134.8, 130.3, 128.7, 127.8, 125.8, 119.6 (q, J_{C-F}=319.3 Hz), 47.4; IR (neat) 3312, 2340, 1578, 1431, 1369, 1229, 1187, 1140, 1099, 1055, 872, 782, 597 cm⁻¹; HRMS (ESI-TOF) calcd for. C₈H₆ClF₃NO₂S ([M−H]⁻): 271.9765, Found: 271.9771.

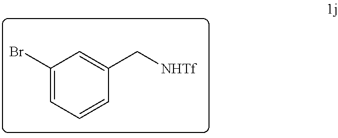

¹H NMR (400 MHz, CDCl₃) δ 7.49-7.46 (m, 2H), 7.26-7.24 (m, 2H), 5.36 (br, 1H), 4.39 (d, J=6.0 Hz, 2H); ¹³C NMR (100 MHz, CDCl₃) δ 137.4, 131.6, 130.7, 130.5, 126.3, 122.8, 119.5 (q, J_{C-F}=319.3 Hz), 47.3; IR (neat) 3314, 1573, 1430, 1370, 1229, 1190, 1142, 1056, 852, 780, 597 cm⁻¹; HRMS (ESI-TOF) calcd. for C₈H₆BrF₃NO₂S ([M−H]⁻): 315.9260, Found: 315.9271.

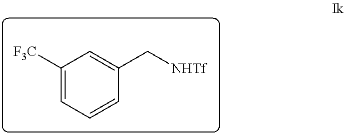

¹H NMR (400 MHz, CDCl₃) δ 7.64-7.52 (m, 4H), 5.20 (br, 1H), 4.52 (d, J=6.0 Hz, 2H); ¹³C NMR (100 MHz, CDCl₃) δ 136.3, 131.5 (q, J_{C-F}=32.5 Hz), 131.1, 129.7, 125.5 (q, J_{C-F}=3.7 Hz), 124.5 (q, J_{C-F}=3.7 Hz), 123.7 (q, J_{C-F}=270.8 Hz), 119.6 (q, J_{C-F}=319.2 Hz), 47.6; IR (neat) 3310, 2924, 1432, 1371, 1328, 1193, 1122, 1071, 879, 799, 606 cm⁻¹; HRMS (ESI-TOF) calcd. for C₉H₆CF₆NO₂S ([M−H]⁻): 306.0029, Found: 306.0034.

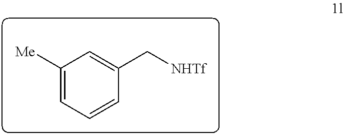

¹NMR (400 MHz, CDCl₃) δ 7.26 (t, J=7.2 Hz, 1H), 7.16-7.09 (m, 3H), 5.09 (br, 1H), 4.38 (d, J=5.6 Hz, 2H), 2.36 (s, 3H); ¹³C NMR (100 MHz, CDCl₃) δ 138.8, 135.1, 129.2, 128.8, 128.5, 124.8, 119.7 (q, J_{C-F}=319.1 Hz), 48.0, 21.1; IR (neat) 3313, 2925, 2334, 1612, 1427, 1370, 1229, 1188, 1142, 1051, 852, 783, 697, 596 cm⁻¹; HRMS (ESI-TOF) calcd. for C₉H₉F₃NO₂S ([M−H]⁻): 252.0312, Found: 252.0314.

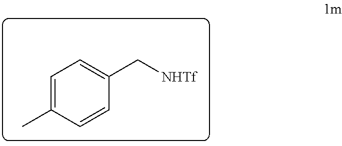

¹H NMR (400 MHz, CDCl₃) δ 7.25-7.15 (m, 4H), 4.92 (br, 1H), 4.40 (d, J=6.0 Hz, 2H), 2.36 (s, 3H); ¹³C NMR (100 MHz, CDCl₃) δ 138.6, 132.1, 129.7, 127.8, 119.7 (q, J_{C-F}=319.4 Hz), 48.0, 21.1; IR (neat) 3314, 2927, 1517, 1428, 1370, 1230, 1189, 1143, 1049, 861, 807, 608 cm⁻¹; HRMS (ESI-TOF) calcd. for C₉H₉F₃NO₂S ([M−H]⁻): 252.0312, Found: 252.0310.

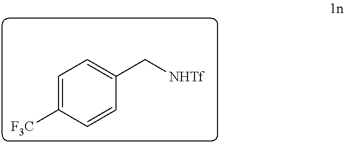

¹H NMR (400 MHz, CDCl₃) δ 7.67 (d, J=8.0 Hz, 2H), 7.47 (d, J=8.0 Hz, 2H), 5.12 (br, 1H), 4.53 (d, J=6.0 Hz, 2H); ¹³C NMR (100 MHz, CDCl₃) δ 139.1, 130.9 (q, J_{C-F}=32.5 Hz), 128.0, 126.0 (q, J_{C-F}=3.8 Hz), 123.8 (q, J_{C-F}=270.8 Hz), 119.6 (q, J_{C-F}=319.1 Hz), 47.6; IR (neat) 3312, 2925, 1622, 1430, 1372, 1324, 1193, 1167, 1125, 1065, 1018, 864, 821, 601 cm^{-1;} HRMS (ESI-TOF) calcd. for C₉H₆F₆NO₂S ([M−H]⁻): 306.0029, Found: 306.0028.

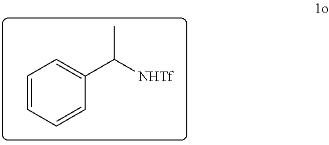

¹H NMR (400 MHz, CDCl₃) δ 7.41-7.30 (m, 5H), 5.13 (br, 1H), 4.80 (dq, J₁=J₂=7.2 Hz, 1H), 1.64 (d, J=6.8 Hz, 3H); ¹³C NMR (100 MHz, CDCl₃) δ 141.1, 128.9, 128.1, 125.8, 119.5 (q, J_{C-F}=319.2 Hz), 55.3, 23.3; IR (neat) 3303, 2985, 2359, 1496, 1430, 1369, 1229, 1189, 1145, 1080, 1022, 979, 868, 761, 698, 623, 592 cm⁻¹; HRMS (ESI-TOF) calcd. for C₉H₉F₃NO₂S ([M−H]⁻): 252.0312, Found: 252.0315.

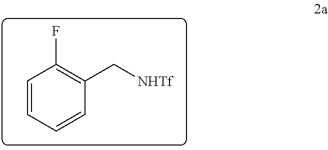

¹H NMR (400 MHz, CDCl₃) δ 7.39-7.34 (m, 211), 7.18 (dt, J=7.6, 1.2 Hz, 1H), 7.13-7.08 (m, 1H), 5.12 (br, 1H), 4.51 (s, 2H); ¹³C NMR (100 MHz, CDCl₃) δ 160.9 (d, J_{C-F}=245.8 Hz), 130.7 (d, J_{C-F}=8.2 Hz), 130.1 (d, J_{C-F}=3.6 Hz), 124.7 (d, J_{C-F}=3.6 Hz), 122.6 (d, J_{C-F}=14.5 Hz), 119.5 (q, J_{C-F}=319.2 Hz), 115.7 (d, J_{C-F}=20.8 Hz), 42.4 (d, J_{C-F}=3.9 Hz); ¹⁹F NMR (376 MHz, CDCl₃) δ-77.6, -118.8; IR (neat) 3316, 2925, 2360, 2340, 1620, 1590, 1494, 1433, 1373, 1230, 1190, 1143, 1108, 1052, 860, 757, 612 cm⁻¹; HRMS (ESI-TOF) calcd. for C₈H₆F₄NO₂S ([M−H]⁻): 256.0061, Found: 256.0060.

### Example 2

### General Procedure of Pd(OTf)₂-Catalyzed Ortho-Fluorination

In a 20 mL sealed tube, benzylamine triflamides 1 (0.2 mmol, 1.0 equiv.), Pd(OTf)₂2H₂O (8.8 mg, 0.02 mmol, 0.1 equiv.), N-fluoro-2,4,6-trimethylpyridinium triflate 10 (1.5 equiv. for 1b-1h and 2a, 2.0 equiv. for 1i-1l and la for mono-fluorination, 3.0 equiv. for 1a and 1m-1h for di-fluorination), NMP (10 μL, 0.1 mmol, 0.5 equiv.) [or DMF (0.5 equiv.) for 1a and 1l for mono-fluorination)] were dissolved in 0.5 mL dry DCE (or PhCF₃ for 1a and 1m-1h for di-fluorination) under air. The tube was sealed with a Teflon lined cap and the reaction mixture was stirred at 120° C. for the given time in Tables 1-2. After cooling to room temperature, the mixture was concentrated under vacuum and the residue was purified by column chromatography on silica gel with a gradient eluant of hexane and ethyl acetate afforded the product 2.

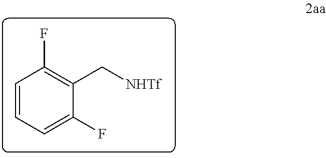

¹H NMR (400 MHz, CDCl₃) δ 7.39-7.31 (m, 1H), 6.99-6.93 (m, 2H), 5.27 (br, 1H), 4.58 (d, J=5.6 Hz, 2H); ¹³C NMR (100 MHz, CDCl₃) δ 161.1 (dd, J_{C-F}=248.8, 7.3 Hz), 131.0 (t, J_{C-F}=10.4 Hz), 119.5 (q, J_{C-F}=319.2 Hz), 117.3 (t, J_{C-F}=18.8 Hz), 117.2 (dd, J_{C-F}=19.0, 6.0 Hz), 36.0 (t, J_{C-F}=3.9 Hz); ¹⁹F NMR (376 MHz, CDCl₃) δ-77.7, −115.2; IR (neat) 3312, 2922, 2360, 1628, 1594, 1472, 1436, 1374, 1230, 1190, 1141, 1046, 929, 849, 784, 605 cm⁻¹; HRMS (ESI-TOF) calcd. for C₈H₅F₅NO₂S ([M−H]⁻): 273.9967, Found: 273.9964.

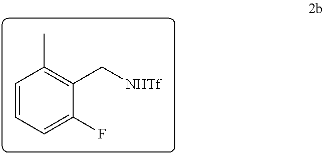

¹NMR (400 MHz, CDCl₃) δ 7.27-7.21 (m, 1H), 7.02 (d, J=7.6 Hz, 1H), 6.95 (d, J=8.8 Hz, 1H), 5.14 (br, 1H), 4.52 (d, J=5.6 Hz, 2H), 2.42 (s, 3H); ¹³C NMR (100 MHz, CDCl₃) δ 161.7 (d, J_{C-F}=244.6 Hz), 139.2 (d, J_{C-F}=3.0 Hz), 130.2 (d, J_{C-f}=9.5 Hz), 126.5 (d, J_{C-F}=3.0 Hz), 120.8 (d, J_{C-F}=14.0 Hz), 119.6 (q, J_{C-F}=319.5 Hz), 113.2 (d, J_{C-F}=22.0 Hz), 39.1 (d, J_{C-F}=5.1 Hz), 18.7 (d, J_{C-F}=2.6 Hz); ¹⁹F NMR (376 MHz, CDCl₃) δ-77.4, −118.3; IR (neat) 3314, 2926, 2360, 1619, 1585, 1429, 1230, 1190, 1143, 1046, 911, 854, 781, 608 cm⁻¹; HRMS (ESI-TOF) calcd. for C₉H₈F₄NO₂S ([M−H]⁻): 270.0217, Found: 270.0221.

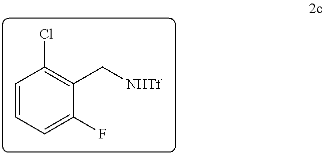

¹H NMR (400 MHz, CDCl₃) δ 7.25 (dt, J=8.4, 6.0 Hz, 1H), 7.18 (d, J=6.8 Hz, 1H), 7.00 (dt, J=9.2, 1.2 Hz, 1H), 5.27 (br, 1H), 4.59 (s, 2H); ¹³C NMR (100 MHz, CDCl₃) δ 161.3 (d, J_{C-F}=250.1 Hz), 135.2 (d, J_{C-F}=4.7 Hz), 131.0 (d, J_{C-F}=9.7 Hz). 125.7 (d, J_{C-F}=3.6 Hz), 121.5 (d, J_{C-F}=17.3 Hz), 119.8 (q, J_{C-F}=319.3 Hz), 114.6 (d, J_{C-F}=22.3 Hz), 39.2 (d, J_{C-F}=4.2 Hz); ¹⁹F NMR (376 MHz, CDCl₃) δ-77.7, −113.3; IR (neat) 3312, 2357, 1608, 1582, 1456, 1433, 1377, 1231, 1195, 1143, 1051, 880, 783, 606 cm⁻¹; HRMS (ESI-TOF) calcd. for C₈H₅ClF₄NO₂S ([M−H]⁻): 289.9671, Found: 289.9678.

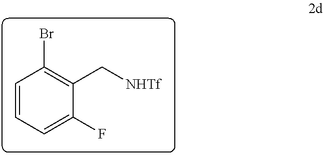

¹H NMR (400 MHz, CDCl₃) δ 7.34 (d, J=8.4 Hz, 1H), 7.21-7.15 (m, 1H), 7.03 (t, J=8.8 Hz, 1H), 5.44 (br, 1H), 4.59 (s, 2H); ¹³C NMR (100 MHz, CDCl₃) δ 161.2 (d, J_{C-F}=251.3 Hz), 131.5 (d, J_{C-F}=9.4 Hz), 128.9 (d, J_{C-F}=3.6 Hz), 124.9 (d, J_{C-F}=3.8 Hz), 123.1 (d, J_{C-F}=17.1 Hz), 119.5 (q, J_{C-F}=319.5 Hz), 115.3 (d, J_{C-F}=22.5 Hz), 41.5 (d, J_{C-F}=4.0 Hz); ¹⁹F NMR (376 MHz, CDCl₃) δ-77.6, −111.9; IR (neat) 3311, 2360, 1605, 1577, 1452, 1431, 1376, 1230, 1194, 1144, 1050, 866, 781, 604 cm⁻¹; HRMS (ESI-TOF) calcd. for C₈H₅BrF₄NO₂S ([M−H]⁻): 333.9166, Found: 333.9171.

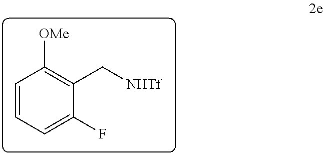

¹H NMR (400 MHz, CDCl₃) δ 7.25-7.19 (m, 1H), 6.67 (t, J=8.8 Hz, 1H), 6.64 (d, J=8.4 Hz, 1H), 5.42 (br, 1H), 4.45 (s, 2H), 3.83 (s, 3H); ¹³C NMR (100 MHz, CDCl₃) δ 160.7 (d, J_{C-F}=246.2 Hz), 158.7 (d, J_{C-F}=7.0 Hz), 130.5 (d, J_{C-F}=10.6 Hz), 119.6 (q, J_{C-F}=319.4 Hz), 111.5 (d, J_{C-F}=17.8 Hz), 108.4 (d, J_{C-F}=22.5 Hz), 106.3 (d, J_{C-F}=3.1 Hz), 56.1, 36.9 (d, J_{C-F}=5.6 Hz); ¹⁹F NMR (376 MHz, CDCl₃) δ-77.8, −117.0; IR (neat) 3314, 2927, 2358, 1619, 1590, 1475, 1423, 1373, 1229, 1188, 1143, 1094, 1048, 910, 858, 778, 609 cm⁻¹; HRMS (ESI-TOF) calcd. for C₉H₈F₄NO₃S ([M−H]⁻): 286.0166, Found: 286.0163.

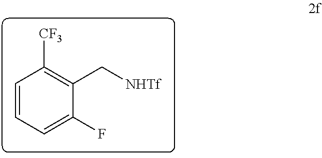

¹H NMR (400 MHz, CDCl₃) δ 7.56-7.50 (m, 2H), 7.41-7.36 (m, 1H), 5.17 (br, 1H), 4.68 (d, J=5.2 Hz, 2H); ¹³C NMR (100 MHz, CDCl₃) δ 161.8 (d, J_{C-F}=249.5 Hz), 131.1 (d, J_{C-F}=9.2 Hz), 130.8 (dq, J_{C-F}=31.0, 3.2 Hz), 123.3 (dq, J_{C-F}=272.5, 3.6 Hz), 122.3 (dq, J_{C-F}=5.5, 3.5 Hz), 120.9 (d, J_{C-F}=16.2 Hz), 120.1 (d, J_{C-F}=21.7 Hz), 119.5 (q, J_{C-F}=319.3 Hz), 38.3 (dq, J_{C-F1}=J_{C-F2}=2.3 Hz); ¹⁹F NMR (376 MHz, CDCl₃) δ-58.9, −77.5, −112.8; IR (neat) 3314, 2923, 2853, 2358, 1592, 1468, 1436, 1378, 1318, 1232, 1185, 1118, 1053, 1048, 999, 889, 801, 726, 602 cm⁻¹; HRMS (ESI-TOF) calcd. for C₉H₅F₇NO₂S ([M−H]⁻): 323.9935, Found: 323.9933.

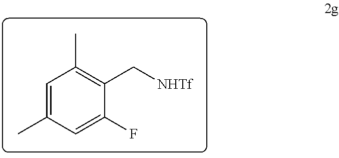

¹H NMR (400 MHz, CDCl₃) δ 6.83 (s, 1H), 6.76 (d, J=8.8 Hz, 1H), 5.07 (br, 1H), 4.47 (d, J=4.4 Hz, 2H), 2.37 (s, 3H), 2.31 (s, 314); ¹³C NMR (125 MHz, CDCl₃) δ 161.6 (d, J_{C-F}=244.0 Hz), 140.9 (d, J_{C-F}=9.4 Hz), 138.7 (d, J_{C-F}=3.4 Hz), 127.2 (d, J_{C-F}=2.8 Hz), 119.6 (q, J_{C-F}=319.4 Hz), 117.6 (d, J_{C-F}=14.0 Hz), 113.7 (d, J_{C-F}=21.8 Hz), 39.0 (d, J_{C-F}=5.0 Hz), 21.1 (d, J_{C-F}=1.9 Hz), 18.7 (d, J_{C-F}=2.8 Hz); ¹⁹F NMR (376 MHz, CDCl₃) δ-77.5, −119.4; IR (neat) 3325, 2928, 2360, 1625, 1578, 1495, 1420, 1373, 1302, 1227, 1184, 1142, 1041, 951, 849, 797, 612 cm⁻¹; HRMS (ESI-TOF) calcd. for C₁₀H₁₀F₄NO₂S ([M−H]⁻): 284.0374, Found: 284.0377.

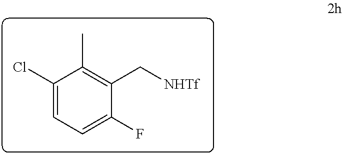

¹H NMR (400 MHz, CDCl₃) δ 7.36 (dd, J=8.8, 5.2 Hz, 1H), 6.93 (t, J=8.8 Hz, 1H), 5.20 (br, 1H), 4.55 (d, J=4.8 Hz, 214), 2.46 (s, 3H); ¹³C NMR (100 MHz, CDCl₃) δ 160.0 (d, J_{C-F}=245.0 Hz), 137.1 (d, J_{C-F}=3.0 Hz), 130.9 (d, J_{C-F}=9.2 Hz), 130.6 (d, J_{C-F}=3.1 Hz), 122.5 (d, J_{C-F}=15.0 Hz), 119.5 (q, J_{C- F}=319.5 Hz), 114.1 (d, J_{C-F}=23.8 Hz), 39.5 (d, J_{C-F}=5.0 Hz), 16.2 (d, J_{C-F}=2.0 Hz); ¹⁹F NMR (376 MHz, CDCl₃) δ-77.4, −118.6; IR (neat) 3309, 2926, 2334, 1609, 1581, 1461, 1435, 1373, 1231, 1187, 1144, 1050, 934, 847, 814, 607 cm⁻¹; HRMS (ESI-TOF) calcd. for C₉H₇ClF₄NO₂S ([M−H]⁻): 303.9828, Found: 303.9833.

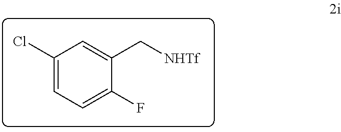

¹H NMR (400 MHz, CDCl₃) δ 7.37-7.31 (m, 2H), 7.06 (t, J=8.8 Hz, 1H), 5.23 (br, 1H), 4.47 (d, J=4.8 Hz, 2H); ¹³C NMR (100 MHz, CDCl₃) δ 159.3 (d, J_{C-F}=245.6 Hz), 130.5 (d, J_{C-F}=8.3 Hz), 129.9 (d, J_{C-F}=3.7 Hz), 124.5 (d, J_{C-F}=16.1 Hz), 119.5 (q, J_{C-F}=319.1 Hz), 117.1 (d, J_{C-F}=22.7 Hz), 42.0 (d, J_{C-F}=3.6 Hz); ¹⁹F NMR (376 MHz, CDCl₃) δ-77.5, −121.4; IR (neat) 3313, 2919, 1489, 1434, 1374, 1231, 1193, 1144, 1116, 1058, 886, 819, 603 cm⁻¹; HRMS (ESI-TOF) calcd. for C₈H₅ClF₄NO₂S ([M−H]⁻): 289.9671, Found: 289.9676.

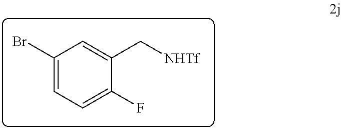

¹H NMR (400 MHz, CDCl₃) δ 7.51-7.45 (m, 2H), 7.01 (t, J=9.2 Hz, 1H), 5.30 (br, 1H), 4.47 (s, 2H); ¹³C NMR (100 MHz, CDCl₃) δ 159.9 (d, J_{C-F}=246.5 Hz), 133.6 (d, J_{C-F}=8.4 Hz), 132.8 (d, J_{C-F}=5.6 Hz), 124.8 (d, J_{C-F}=15.8 Hz), 119.5 (q, J_{C-F}=319.2 Hz), 117.6 (d, J_{C-F}=22.5 Hz), 117.1 (d, J_{C-F}=3.5 Hz), 41.9 (d, J_{C-F}=3.6 Hz); ¹⁹F NMR (376 MHz, CDCl₃) δ-77.5, −120.8; IR (neat) 3313, 2925, 2360, 1485, 1435, 1375, 1231, 1195, 1145, 1117, 1058, 878, 818, 605 cm⁻¹; HRMS (ESI-TOF) calcd. for C₈H₅BrF₄NO₂S ([M−H]⁻): 333.9166, Found: 333.9167.

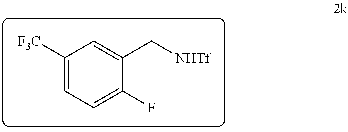

¹H NMR (400 MHz, CDCl₃) δ 7.67-7.64 (m, 2H), 7.26-7.22 (m, 1H), 4.55 (s, 2H); ¹³C NMR (125 MHz, CDCl₃) δ 162.6 (d, J_{C-F}=251.8 Hz), 128.2 (dq, J_{C-F}=9.5, 3.6 Hz), 127.6 (dq, J_{C-F}=J_{C-F}=3.9 Hz), 127.5 (dq, J_{C-F}=33.1, 3.5 Hz), 123.9 (d, J_{C-F}=15.5 Hz), 123.3 (q, J_{C-F}=270.4 Hz), 119.5 (q, J_{C-F}=318.9 Hz), 116.5 (d, J_{C-F}=22.2 Hz), 42.0 (d, J_{C-F}=3.5 Hz); ¹⁹F NMR (376 MHz, CDCl₃) δ-62.4, −77.6, −113.0; IR (neat) 3310, 2924, 2360, 1627, 1607, 1509, 1432, 1373, 1330, 1230, 1171, 1118, 1072, 983, 901, 831, 606 cm⁻¹; HRMS (ESI-TOF) calcd. for C₉H₅F₇NO₂S ([M−H]⁻): 323.9935, Found: 323.9935.

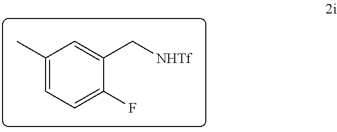

¹H NMR (400 MHz, CDCl₃) δ 7.15-7.12 (m, 2H), 7.00-6.96 (m, 1H), 5.11 (br, 1H), 4.46 (s, 2H), 2.33 (s, 3H); ¹³C NMR (100 MHz, CDCl₃) δ 159.1 (d, J_{C-F}=242.9 Hz), 134.4 (d, J_{C-F}=3.7 Hz), 131.0 (d, J_{C-F}=8.0 Hz), 130.5 (d, J_{C-F}=3.5 Hz), 122.1 (d, J_{C-F}=14.5 Hz), 119.6 (q, J_{C-F}=319.3 Hz), 115.4 (d, J_{C-F}=20.9 Hz), 42.6 (d, J_{C-F}=3.7 Hz), 20.6; ¹⁹F NMR (376 MHz, CDCl₃) δ-77.6, −124.3; IR (neat) 3311, 2918, 2850, 2360, 1751, 1619, 1503, 1433, 1372, 1229, 1188, 1141, 1117, 1048, 911, 887, 815, 598 cm⁻¹; HRMS (ESI-TOF) calcd. for C₉H₈F₄NO₂S ([M−H]⁻): 270.0217, Found: 270.0221.

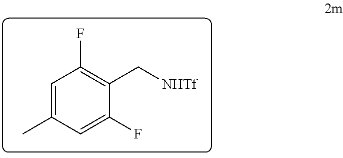

¹H NMR (400 MHz, CDCl₃) δ 6.77 (d, J=8.4 Hz, 2H), 5.16 (br, 1H), 4.53 (s, 2H), 2.36 (s, 3H); ¹³C NMR (100 MHz, CDCl₃) δ 160.8 (dd, J_{C-F}=247.8, 8.2 Hz), 142.4 (t, J_{C-F}=10.0 Hz), 119.5 (q, J_{C-F}=319.2 Hz), 117.2 (dd, J_{C-F}=18.6, 6.0 Hz), 108.5 (t, J_{C-F}=19.1 Hz), 35.9 (t, J_{C-F}=3.7 Hz), 21.4 (t, J_{C-F}=1.9 Hz); ¹⁹F NMR (376 MHz, CDCl₃) δ-77.8, −116.6; IR (neat) 3312, 2923, 2360, 1641, 1587, 1433, 1374, 1230, 1190, 1143, 1053, 936, 841, 609 cm⁻¹; HRMS (ESI-TOF) calcd. for C₉H₇F₅NO₂S ([M−H]⁻): 288.0123, Found: 288.0127.

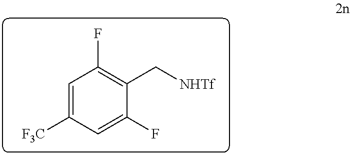

Reaction conditions: PhCF₃ as solvent, 20 mol % Pd(OTf)₂.2H₂O as catalyst, microwave (300 W), 150° C., 2 h. 2n was isolated as a mixture with mono-fluorination product (Di/Mono=9.5/1 by ¹H NMR, and the yield was calculated using this ratio). ¹H NMR (400 MHz, CDCl₃) δ 7.29-7.24 (m, 2H), 4.61 (s, 2H); ¹³C NMR (100 MHz, CDCl₃) δ 161.0 (dd, J_{C-F}=251.6, 7.5 Hz), 133.7 (tq, J_{C-F}=34.8, 10.1 Hz), 122.3 (tq, J_{C-F}=271.5, 3.4 Hz), 119.4 (q, J_{C-F}=319.1 Hz), 115.7 (t, J_{C-F}=18.3 Hz), 109.5 (dm, J_{C-F}=19.9 Hz), 35.7 (t, J_{C-F1}=3.4 Hz); ¹⁹F NMR (376 MHz, CDCl₃) δ-63.6, −77.7, −111.4; IR (neat) 3317, 2363, 1596, 1441, 1358, 1332, 1232, 1195, 1140-1068, 947, 912, 868, 608 cm⁻¹; HRMS (ESI-TOF) calcd. for C₉H4F₈NO₂S ([M−H]⁻): 341.9840, Found: 341.9842.

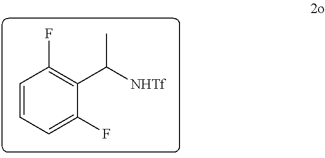

¹H NMR (400 MHz, CDCl₃) δ 7.33-7.26 (m, 1H), 6.97-6.91 (m, 2H), 5.57 (br, 1H), 5.21 (q, J=7.2 Hz, 1H), 1.67 (d, J=7.2 Hz, 3H); ¹³C NMR (100 MHz, CDCl₃) δ 160.1 (dd, J_{C-F}=246.7, 7.8 Hz), 130.0 (t, J_{C-F}=10.7 Hz), 119.4 (q, J_{C-F}=319.1 Hz), 117.3 (t, J_{C-F}=17.6 Hz), 112.0 (dd, J_{C-F}=19.3, 6.2 Hz), 46.3 (t, J_{C-F}=3.0 Hz), 22.7; ¹⁹F NMR (376 MHz, CDCl₃) δ-78.2, −116.3; IR (neat) 3310, 2926, 2360, 1747, 1628, 1593, 1473, 1435, 1379, 1232, 1194, 1147, 1085, 1034, 997, 964, 789, 614 cm⁻¹; HRMS (ESI-TOF) calcd. for C₉H₇F₅NO₂S ([M−H]⁻): 288.0123, Found: 288.0130.

### Example 3

### Transformations of Triflamides

Synthesis of 16:

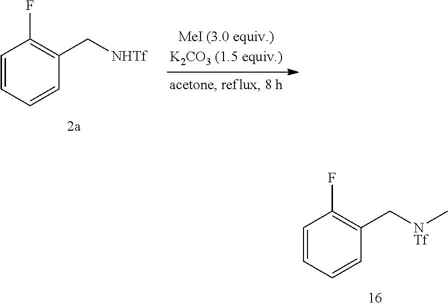

To a stirred solution of 2a (514.4 mg, 2.0 mmol, 1.0 equiv.) in acetone (10 mL) was added K₂CO₃ (414.7 mg, 3.0 mmol, 1.5 equiv.) and then MeI (373.5 μL, 6.0 mmol, 3.0 equiv.) dropwise at room temperature (r.t.). The reaction mixture was heated to reflux and stirred for 8 h. After cooling to r.t., acetone was removed under vacuum and water (10 mL) was added to the residue, extracted with diethyl ether (15 mL×3), dried over Na₂SO₄ and concentrated under vacuum. Purification of the residue by column chromatography on silica gel (hexane-ethyl acetate=50/1 as eluant) afforded 16 (500 mg, 92% yield) as colorless oil. ¹H NMR (400 MHz, CDCl₃) δ 7.43 (dt, J=7.6, 1.6 Hz, 1H), 7.39-7.33 (m, 1H), 7.21 (dt, J=7.6, 1.2 Hz, 1H), 7.13-7.08 (m, 1H), 4.57 (br, 2H), 2.96 (s, 3H); ¹³C NMR (100 MHz, CDCl₃) δ 161.0 (d, J_{C-F}=246.0 Hz), 130.6, 130.5 (d, J_{C-F}=5.0 Hz), 124.8 (d, J_{C-F}=3.6 Hz), 121.2 (d, J_{C-F}=14.0 Hz), 120.2 (q, J_{C-F}=321.6 Hz), 115.7 (d, J_{C-F}=21.4 Hz), 47.5 (d, J_{C-F}=4.2 Hz), 34.9; ¹⁹F NMR (376 MHz, CDCl₃) δ-75.1, −119.1; IR (neat) 2957, 2361, 2339, 1619, 1589, 1493, 1457, 1388, 1338, 1227, 1183, 1150, 1119, 988, 925, 759, 590 cm⁻¹; HRMS (ESI-TOF) calcd. for C₉H₈F₄NO₂S ([M−H]⁻): 270.0217, Found: 270.0213.

### Example 4

### Synthesis of 2-Fluoro-N-methylbenzylamine 11

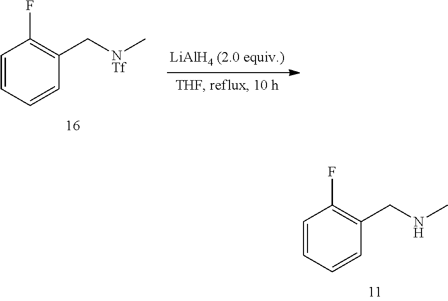

To a stirred solution of 16 (135.6 mg, 0.5 mmol, 1.0 equiv.) in dry THF (5 mL) was added LiAlH₄ (38 mg, 1.0 mmol, 2.0 equiv.) at 0° C. under nitrogen. The reaction mixture was then heated to reflux and stirred for 10 h. After cooling to r.t., the reaction mixture was quenched with water and extracted with diethyl ether (10 mL×3). 2-Fluoro-N-methylbenzylamine 11 was isolated by acid/basic extraction, using 2 N HCl and 2 N NaOH, and evaporation under vacuum (15 Ton) as colorless oil (60 mg, 86% yield). ¹H NMR (400 MHz, CDCl₃) δ 7.32 (dt, J=7.6, 1.6 Hz, 1H), 7.26-7.21 (m, 1H), 7.10 (dt, J=7.6, 1.2 Hz, 1H), 7.06-7.01 (m, 1H), 3.81 (s, 2H), 2.45 (s, 3H).

### Example 5

### Synthesis of 2-Fluorobenzaldehyde 12

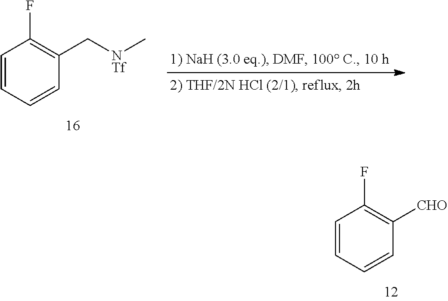

To a stirred solution of 16 (135.6 mg, 0.5 mmol, 1.0 equiv.) in dry DMF (3 mL) was added NaH (36 mg, 1.5 mmol, 3.0 equiv.) at r.t. under nitrogen. The reaction mixture was heated to 100° C. and stirred for 10 h. After cooling to r.t, the reaction mixture was added THF/2 N HCl (2/1, 4 mL) and then heated to reflux for 2 h under nitrogen. The solution was cooled to r.t. again, diluted with diethyl ether (25 mL), and washed with water (10 mL×3). The yield (86%) of aldehyde 12 was determined by GC [Shimadzu GCMS-QP2010S equipped with a SHRXI-5MS column (30 m, 0.25 mm ID, 0.25 μm DF)] with p-NO₂-benzaldehyde as internal standard. ¹H NMR (400 MHz, CDCl₃) δ 10.38 (d, J=0.8 Hz, 1H), 7.88 (dt, J=7.6, 2.0 Hz, 1H), 7.64-7.58 (m, 1H), 7.30-7.26 (m, 1H), 7.20-7.15 (m, 1H).

### Example 6

### General Procedure for Nucleophilic Substitution

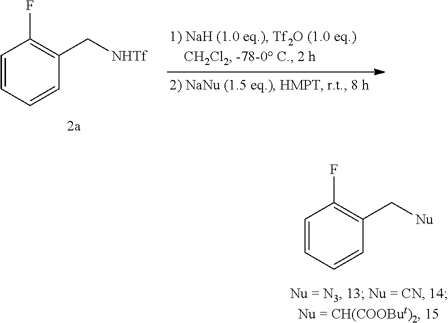

To a stirred solution of 2a (135.6 mg, 0.5 mmol, 1.0 equiv.) in dry dichloromethane (3 mL) was added NaH (12 mg, 0.5 mmol, 1.0 equiv.) at −78° C. under nitrogen. After stirring for 5 minutes at that temperature, trifluoromethanesulfonic anhydride (84.2 μL, 0.5 mmol, 1.0 equiv.) was added dropwise. The reaction mixture was stirred at −78° C. for 1.5 h and then 0.5 h at 0° C., quenched by ice water, extracted with dichloromethane (10 mL×3). The solvent was removed under vacuum (about 10-15° C.) and the residue was dried with an oil pump for several minutes. The residue was then dissolved in HMPT (3 mL), and NaNu (0.75 mmol, 1.5 equiv.) was added in one portion when the reaction mixture was cooled by ice water. After stirring for 8 h at r.t., the reaction mixture was diluted with water (5 mL) at 0° C., extracted with diethyl ether (10 mL×3) and then the combined organic phase was washed with water (10 mL×3). Evaporation and column chromatography on silica gel afforded 13-15 all as a colorless oil.

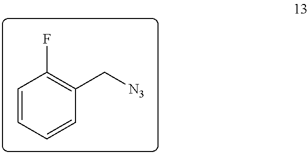

70% yield; ¹H NMR (400 MHz, CDCl₃) δ 7.36-7.31 (m, 2H), 7.17 (dt, J=7.6, 1.2 Hz, 1H), 7.13-7.08 (m, 1H), 4.41 (s, 2H); ¹³C NMR (100 MHz, CDCl₃) δ 160.8 (d, J_{C-F}=246.4 Hz), 130.3 (d, J_{C-F}=3.8 Hz), 130.1 (d, J_{C-F}=8.1 Hz), 124.3 (d, J_{C-F}=3.7 Hz), 122.6 (d, J_{C-F}=15.1 Hz), 115.5 (d, J_{C-F}=21.1 Hz), 48.3 (d, J_{C-F}=3.4 Hz); ¹⁹F NMR (376 MHz, CDCl₃) δ-118.2; IR (neat) 2930, 2095, 1618, 1588, 1491, 1454, 1349, 1234, 1179, 1105, 1034, 884, 840, 755, 670 cm⁻¹.

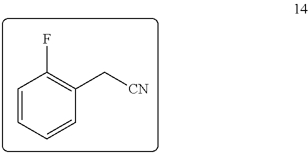

68% yield; ¹H NMR (400 MHz, CDCl₃) δ 7.44 (t, J=7.6 Hz, 1H), 7.37-7.31 (m, 1H), 7.18 (t, J=7.6 Hz, 1H), 7.10 (t, J=9.2 Hz, 1H), 3.77 (s, 2H).

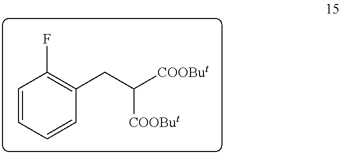

65% yield; NaCH(COOBu’)₂ was generated from NaH (1.5 mmol) and CH₂(COOBA (1.5 mmol) in situ in HMPT (0.5 mL). ¹HNMR (400 MHz, CDCl₃) δ 7.22-7.15 (m, 2H), 7.04-6.97 (m, 2H), 3.56 (t, J=8.0 Hz, 1H), 3.15 (d, J=8.0 Hz, 2H), 1.39 (s, 18H); ¹³C NMR (100 MHz, CDCl₃) δ 168.0, 161.3 (d, J_{C-F}=244.2 Hz), 131.4 (d, J_{C-F}=4.7 Hz), 128.4 (d, J_{C-F}=8.1 Hz), 125.1 (d, J_{C-F}=15.3 Hz), 123.8 (d, J_{C-F}=3.4 Hz), 115.2 (d, J_{C-F}=21.7 Hz), 81.5, 53.7 (d, J_{C-F}=1.5 Hz), 28.3 (d, J_{C-F}=2.3 Hz), 27.8; ¹⁹F NMR (376 MHz, CDCl₃) δ-117.7; IR (neat) 2978, 2934, 1725, 1585, 1493, 1455, 1393, 1368, 1243, 1134, 1103, 1060, 1033, 996, 846, 755 cm⁻¹. HRMS (ESI-TOF) calcd. for C₁₈H₂₅FNaO₄ ([M+Na]⁺): 347.1629, Found: 347.1632.

### Example 7

### Versatile Pd(OTf)₂-Catalyzed ortho-Fluorination Using NMP as a Promoter

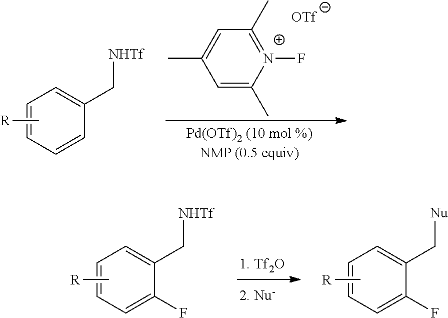

Triflamide directed ortho-palladation was established (Li, J.-J.; Mei, T.-S.; Yu, J.-Q. Angew. Chem., Int. Ed. 2008, 47, 6452). The use of either a mixture of DCE/DMF (20:1) or DCE in the presence of Cs₂CO₃ as the reaction media was found to be important for palladation to occur. However, reaction of la with 10 mol % Pd(OAc)₂ and various fluorine sources previously used by Sanford (3-6) (Hull, K. L.; Anani, W. Q.; Sanford, M. S. J. Am. Chem. Soc. 2006, 128, 7134) and other fluorinating reagents 7 and 8 under similar conditions (various solvents in the presence of mild bases such as DMF and Cs₂CO₃) gave only low to moderate yields of the desired fluorinated products. It was found through screening that the presence of 0.5 equiv of NMP (N-methylpyrrolidinone) in DCE increased the yield to 45% using N-fluoro-2,4,6-trimethylpyridinium tetrafluoroborate (6), while other fluorine sources 3-8 were less effective than this (Table 2).

TABLE 2
NMP-Promoted Fluorination with Fluorine Source

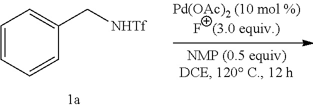

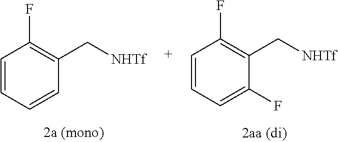

| | |
|---|---|
| 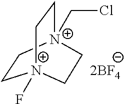 | 3 |
| 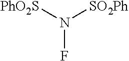 | 4 |
| 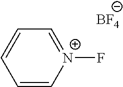 | 5 |
| 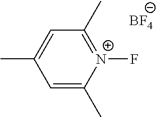 | 6 |
| 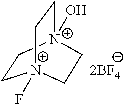 | 7 |
| 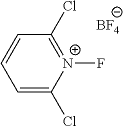 | 8 |
| 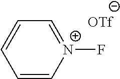 | 9 |
| 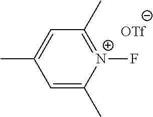 | 10 |

During this screening process, two competing side pathways, acetoxylation and carbonylative lactamization, were noticed to accompany fluorination. The carbonyl source for the carbonylative lactamization likely comes from the decomposition of acetate. These observations led to the testing of whether fluorinating reagents 9 and 10 could either suppress these side reactions or enhance the rate of reductive elimination of fluoride. It was found that the use of N-fluoro-2,4,6-trimethylpyridinium triflate 10 greatly increased the yield to give a mixture of 2a (20%) and 2aa (51%) as products (Table 2). The best results for this combination of substrate, catalyst, fluorine source, and promoter were obtained in DCE, PhCF₃. This improvement by replacing BE₁ with OTf prompted a revisit to this reaction in the absence of acetate anions by using Pd(CH₃CN)₄(OTs)₂, Pd(CH₃CN)₄(OTf)₂, Pd(NTf₂)₂, or Pd(OTf)₂. It was found that the use of any of these catalysts improved the reaction to some extent. The best results were obtained with Pd(NTf₂)₂ or Pd(OTf)₂ to give mainly difluorinated product 2aa in 65% and 68% yield respectively, with a shortening of the reaction time from 12 h to 4 h. The reaction was also sensitive to the quantity of NMP, with 0.5 equiv being optimal for this combination of substrate, catalyst, fluorine source, and promoter. The use of 0.1 or 5 equiv of NMP reduced the yield to 30%.

With this newly established fluorination protocol, ortho-substituted substrates were fluorinated to give the corresponding products in 60-88% yields (see Table 3). Both electron donating (OMe) and withdrawing groups (CF₃, F, Cl, Br) were tolerated (2aa, 2b-h). The use of 5 mol % Pd catalyst was sufficient, albeit requiring longer reaction times (2b and 2c). The presence of Cl and Br in the products is very useful for further synthetic elaborations. Fluorination of meta-substituted arenes gives mono-fluorinated products predominantly (2i-l). Attempts to achieve mono-selectivity with non-substituted benzylamine triflamides were moderately successful. While di-fluorinated products 2aa was obtained in 68% yield (-4% 2a), the best yield of mono-fluorinated product 2a was 41% (˜7% 2aa) when the reaction was performed using 0.5 equiv DMF instead of NMP.

TABLE 3
Pd(OTf)₂-Catalyzed ortho-Fluorination^{a}

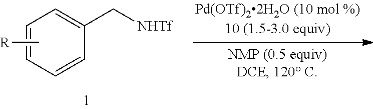

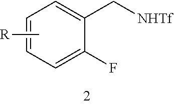

| | |
|---|---|
| 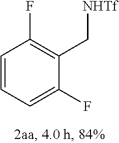 | 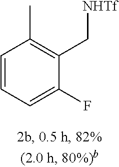 |
| 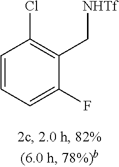 | 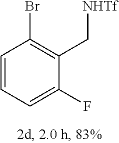 |
| 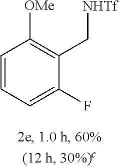 | 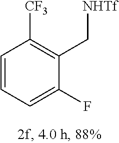 |
| 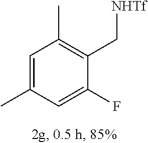 | 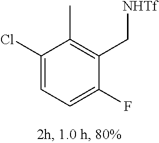 |
| 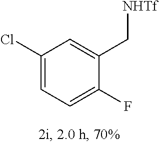 | 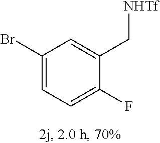 |
| 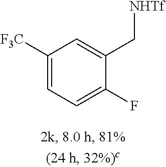 | 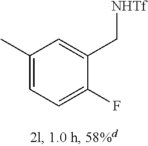 |
| 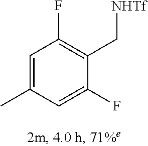 | 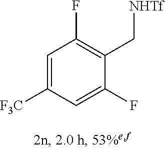 |
| 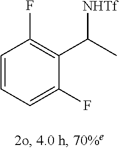 | 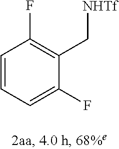 |

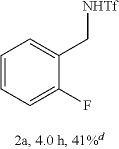

^{a}Isolated yield.
^{b}5 mol % Pd(OTF)₂•2H₂O.
^{c}Pd(OAc)₂ was used instead of Pd(OTf)₂•2H₂O.
^{d}DMF (0.5 equiv) was used instead of NMP.
^{e}PhCF₃ was used as solvent.
^{f}20 mol % Pd(OTf)₂•2H₂O and the reaction was performed at 150 ° C. under microwave heating.

Five major classes of ortho-fluorinated synthons, namely, benzaldehyde, benzylamine, benzylazide, phenylacetonitrile and phenylpropanoate were synthesized from the ortho-fluorinated aryl, thus greatly expanding the scope of ArF synthesis. The ortho-fluorinated phenylpropionic acids are especially valuable as they can not be accessed by either ortho-lithiation or by palladation using previously reported directing groups.

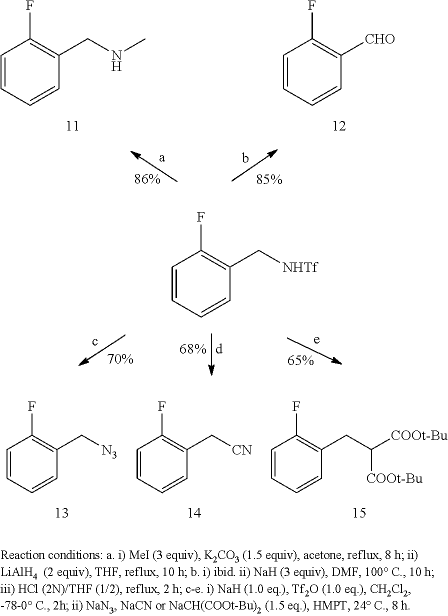

Thus, the present invention provides a new protocol for efficient ortho-fluorination using, for example, Pd(OTf)₂ as the catalyst, N-fluoro-2,4,6-trimethylpyridinium triflate as the fluorine source and NMP as the promoter. The triflamide directing group can be readily displaced by a wide range of heteroatom and carbon nucleophiles, thereby affording this fluorination protocol excellent versatility for synthetic applications.

When ranges are used herein for physical properties, such as molecular weight, or chemical properties, such as chemical formulae, all combinations and subcombinations of ranges and specific embodiments therein are intended to be included.

Although the invention has been illustrated and described with respect to one or more implementations, equivalent alterations and modifications will occur to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

The Abstract of the disclosure will allow the reader to quickly ascertain the nature of the technical disclosure. It is submitted with the understanding that it will not be used to interpret or limit the scope or meaning of the following claims.

1. A method of fluorinating a compound comprising:
reacting a compound having the formula: 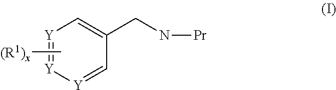
each Y is CR¹, CH, N, O, or S, wherein if Y is N, O, or S, at least one ring atom adjacent to Y is CR¹;
1. each R¹ is independently selected from the group of radicals consisting of C₁₋₂ alkyl, C₂₋₂₀ alkenyl, C₂₋₂₀ alkynyl, C₁-C₂₀ alkoxy, C₆-C₂₀ aryl, C₇-C₂₀ alkylaryl, C₄-C₂₀ heterocycle, C₄-C₂₀ heteroaryl, C₄-C₂₀ alkylheterocycle, C₇-C₂₀ alkylheteroaryl, C₆-C₂₀ aryloxy, —OH, —CO, —COOH, —CN, —N₃, halo, —CF₃, —OCF₃, —NH₂, —NO₂, —NR³R^{3′}, —N(O)R³, —SH, —SR³, —SOR³, —SO₂R³, —C(O)R³, —CO₂R³, —C(O)NR³R^{3′}, and —OC(O)R³; wherein the alkyl, alkenyl, alkynyl, alkoxy, aryl, heterocycle, heteroaryl, or aryloxy may be substituted or unsubstituted and wherein in the alkyl portion one or more: —CH₂—, —CH₂CH₂—, —(CH₂)ₙ— groups are each optionally replaced by —O— or —NH—; wherein n is an integer equal to or greater than 1;
or wherein two R¹ are joined together to form a bicyclic or tricyclic alkyl or aryl with the ring to which they are attached, wherein if the bicyclic or tricyclic alkyl or aryl is a heterocycle, at least one ring atom adjacent to the heteroatom is substituted;
R³ and R^{3′} are each independently selected from the group of radicals consisting of H, C₁₋₂₀ alkyl, C₂₋₂₀ alkenyl, C₂₋₂₀ alkynyl, C₆-C₂₀ aryl, C₇-C₂₀ alkylaryl, C₄-C₂₀ heterocycle, C₄-C₂₀ heteroaryl, C₄-C₂₀ alkylheterocycle, and C₇-C₂₀ alkylheteroaryl; wherein the alkyl, alkenyl, alkynyl, aryl, heterocycle, or heteroaryl, be substituted or unsubstituted and wherein in the alkyl portion one or more —CH₂—, —CH₂CH₂—, ‘3(CH₂)n-, groups are each optionally replaced by —O— or —NH—; n is an integer equal to or greater than 1;
Pr is a protecting group; and
x is 0, 1, 2, 3, or 4;
a palladium (II) catalyst;
a fluorinating reagent; and
a promoter having the formula 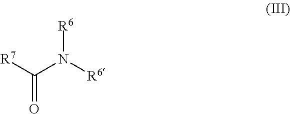
wherein
R⁶ and R^{6′} are each H or a C₁₋₆ alkyl, wherein at least one of R⁶ and R^{6′} is not H,
R⁷ is H or a C₁₋₆ alkyl,
or wherein either R⁶ and R^{6′} together with the nitrogen to which they are attached or R⁶ and R⁷ together with the nitrogen and carbonyl to which they are attached form a 5- or 6-membered ring which may be unsubstituted or substituted with one or more C₁₋₆ alkyl groups;
to form at least one of the ortho-fluorinated compounds having the formulas: 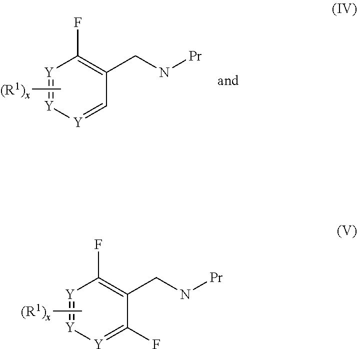
wherein R¹, x, and Pr are described above.

2. The method of claim 1, wherein Pr is a trifluoromethanesulfonyl group.

3. The method of claim 1, wherein x is 0, 1, or 2, and each R¹ is independently selected from the group of radicals consisting of C₁₋₆ alkyl, C₂₋₆ alkenyl, C₂₋₆ alkynyl, C₁-C₆ alkoxy , —OH, —CO, —COOH, —CN, —N₃, halo, —CF₃, —OCF₃, —NH₂, —NO₂, —NR³R^{3′}, —N(O)R³, —SH, —SR³, —SOR³, —SO₂R³, —C(O)R³, —CO₂R³, —C(O)NR³R^{3′}, and —OC(O)R³; wherein the alkyl, alkenyl, alkynyl, or alkoxy, may be substituted or unsubstituted and wherein in the alkyl portion one or more: —CH₂—, —CH₂CH₂—, —(CH₂)ₙ— groups are each optionally replaced by —O— or —NH—; and R³ and R^{3′} are each independently selected from the group of radicals consisting of H, C₁₋₆ alkyl, C₂₋₆ alkenyl, and C₂₋₆ alkynyl; wherein n is an integer equal to or greater than 1.

4. The method of claim 1, wherein the compound of formula (I) has the formula:

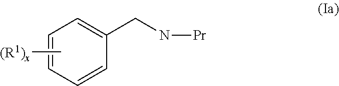

5. The method of claim 1, wherein the palladium (II) catalyst is Pd(CH₃CN)₄(OTs)₂, Pd(CH₃CN)₄(OTf)₂, Pd(NTf₂)₂, or Pd(OTf)₂, wherein Ts is toluenesulfonyl, and Tf is trifluoromethanesulfonyl.

6. The method of claim 5, wherein the palladium (II) catalyst is Pd(NTf₂)₂ or Pd(OTf)₂.

7. The method of claim 1, wherein 5-20 mol % of the palladium (II) catalyst is used.

8. The method of claim 1, wherein the fluorinating agent is a compound having the formula: 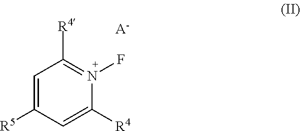
wherein
A⁻ is a counter ion; and
R⁴, R^{4′}, and R⁵ are each independently halogen, C₁₋₁₂ alkyl, or C₂₋₁₂ alkenyl, wherein the alkyl or alkenyl may be substituted with one or more halogen.

9. The method of claim 8, wherein A⁻ is OTf⁻, BF₄⁻, or PF₆⁻.

10. The method of claim 8, wherein the fluorinating agent is a compound having the formula: 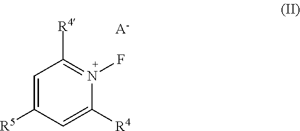
wherein
A⁻ is OTf⁻, BF₄⁻, or PF₆⁻;
R⁴ and R^{4′} are each independently a C₁₋₆ alkyl; and
R⁵ is H or a C₁₋₆ alkyl.

11. The method of claim 10, wherein the fluorinating reagent is N-fluoro-2-4-6-trimethylpyridinium triflate.

12. The method of claim 1, wherein 1-5 equivalents of the fluorinating reagent is used.

13. The method of claim 1, wherein the promoter is N-methylpyrrolidinone (NPM).

14. The method of claim 1, wherein 0.1-5 equivalents of the promoter is used.

15. The method of claim 1, wherein 0.3-0.7 equivalents of the promoter is used.

16. The method of claim 1, wherein the promoter is dimethyl formamide (DMF).

17. The method of claim 1, wherein the solvent is 1,1-dichloroethene (DCE) or PhCF₃.

18. The method of claim 1, wherein the yield of difluorinated product is at least 50%.

19. The method of claim 1, wherein the yield of monofluorinated product is at least 40%.

20. The method of claim 1, wherein microwave heating is not used in the reaction.

21. A method of making a fluorinated compound comprising:
(a) reacting a compound having the formula: 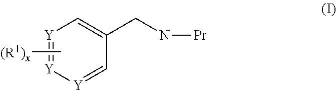
each Y is CR¹, CH, N, O, or S, wherein if Y is N, O, or S, at least one ring atom adjacent to Y is CR¹;
each R¹ is independently selected from the group of radicals consisting of C₁₋₂₀ alkyl, C₂₋₂₀ alkenyl, C₂₋₂₀ alkynyl, C₁-C₂₀ alkoxy, C₆-C₂₀ aryl, C₇-C₂₀ alkylaryl, C₄-C₂₀ heterocycle, C₄-C₂₀ heteroaryl, C₄-C₂₀ alkylheterocycle, C₇-C₂₀ allcylheteroaryl, C₆-C₂₀ aryloxy, —OH, —CO, —COOH, —CN, —N₃, halo, —CF₃, —OCF₃, —NH₂, —NO₂, —NR³R^{3′}, —N(O)R³, —SH, —SR³, —SOR³, —SO₂R³, —C(O)R³, —CO₂R³, —C(O)NR³R^{3′}, and —OC(O)R³; wherein the alkyl, alkenyl, alkynyl, alkoxy, aryl, heterocycle, heteroaryl, or aryloxy may be substituted or unsubstituted and wherein in the alkyl portion one or more: —CH₂—, CH₂CH₂—, —(CH₂)n- groups are each optionally replaced by —O— or —NH—; wherein n is an integer equal to or greater than 1.
or wherein two R¹ are joined together to form a bicyclic or tricyclic alkyl or aryl with the ring to which they are attached, wherein if the bicyclic or tricyclic alkyl or aryl is a heterocycle, at least one ring atom adjacent to the heteroatom is substituted;
R³ and R^{3′} are each independently selected from the group of radicals consisting of H, C₁₋₂₀ alkyl, C₂₋₂₀ alkenyl, C₂₋₂₀ alkynyl, C₆-C₂₀ aryl; C₇-C₂₀ alkylaryl, C₄-C₂₀ heterocycle, C₄-C₂₀ heteroaryl, C₄-C₂₀ alkylheterocycle, and C₇-C₂₀ alkylheteroaryl; wherein the alkyl, alkenyl, alkynyl, aryl, heterocycle, or heteroaryl, be substituted or unsubstituted and wherein in the alkyl portion one or more CH₂—, CH₂CH₂—, —(CH₂)n- —CH₂CH₂— groups are each optionally replaced by —O‘— or —NH—; wherein n is an integer equal to or greater than 1;
Pr is a protecting group; and
x is 0, 1, 2, 3, or 4;
a palladium (II) catalyst;
a fluorinating reagent; and
a promoter having the formula 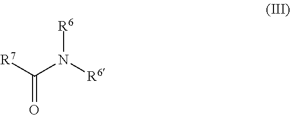
wherein
R⁶ and R^{6′} are each H or a C₁₋₆ alkyl, wherein at least one of R⁶ and R^{6′} is not H,
R⁷ is H or a C₁₋₆ alkyl,
or wherein either R⁶ and R^{6′} together with the nitrogen to which they are attached or R⁶ and R⁷ together with the nitrogen and carbonyl to which they are attached form a 5- or 6-membered ring which may be unsubstituted or substituted with one or more C₁₋₆ alkyl groups;
to form at least one of the ortho-fluorinated compounds having the formulas: 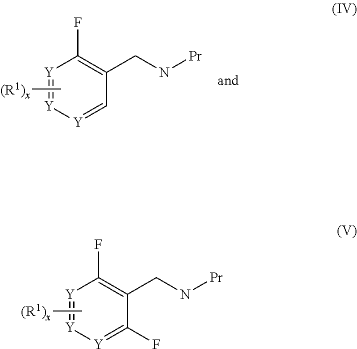
wherein R¹, x, and Pr are described above; and
(b) reacting the compound of formula (IV) or (V) with a reagent to obtain at least one of the compounds having the formula: 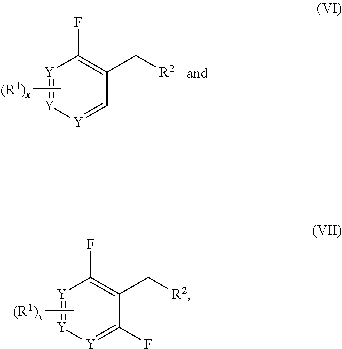
wherein each R² is independently selected from the group of radicals consisting of C₁₋₂₀ alkyl, C₂₋₂₀ alkenyl, C₂₋₂₀ alkynyl, C₁-C₂₀ alkoxy, C₆-C₂₀ aryl, C₇-C₂₀ alkylaryl, C₄-C₂₀ heterocycle, C₄-C₂₀ heteroaryl, C₄-C₂₀ alkylheterocycle, C₇-C₂₀ alkylheteroaryl, C₆-C₂₀ aryloxy, —OH, —CO, —COOH, —CN, —N₃, halo, —CF₃, —OCF₃, —NH₂, —NO₂, —NR³R^{3′}, —N(O)R³, —SH, —SR³, —SOR³, —SO₂R³, —C(O)R³, —CO₂R³, —C(O)NR³R^{3′}, and —OC(O)R³; wherein the alkyl, alkenyl, alkynyl, alkoxy, aryl, heterocycle, heteroaryl, or aryloxy be substituted or unsubstituted and wherein in the alkyl portion one or more —CH₂—, CH₂CH₂—, —(CH₂)n- groups are each optionally replaced by —O— or —NH—; wherein n is an integer equal to or greater than 1; and
R³ and R^{3′} are each independently selected from the group of radicals consisting of H, C₁₋₂₀ alkyl, C₂₋₂₀ alkenyl, C₂₋₂₀ alkynyl, C₆-C₂₀ aryl; C₇-C₂₀ alkylaryl, C₄-C₂₀ heterocycle, C₄-C₂₀ heteroaryl, C₄-C₂₀ alkylheterocycle, and C₇-C₂₀ alkylheteroaryl; wherein the alkyl, alkenyl, allcynyl, aryl, heterocycle, or heteroaryl, be substituted or unsubstituted and wherein in the alkyl portion one or more: —CH₂—, —CH₂CH₂—, —(CH₂)n- groups are each optionally replaced by —O— or —NH—; wherein n is an integer equal to or greater than 1; and
each of R¹, Y, and x are described above.

22. The method of claim 21, wherein Pr is a trifluoromethanesulfonyl group.

23. The method of claim 21, wherein each Y is CH or CR¹.

24. The method of claim 21, wherein the palladium (II) catalyst is Pd(CH₃CN)₄(OTs)₂, Pd(CH₃CN)₄(OTf)₂, Pd(NTf₂)₂, or Pd(OTf)₂, wherein Ts is toluenesulfonyl, and Tf is trifluoromethanesulfonyl.

25. The method of claim 24 wherein the palladium (II) catalyst is Pd(NTf₂)₂ or Pd(OTf)₂.

26. The method of claim 21, wherein the fluorinating agent is a compound having the formula: 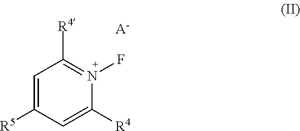
wherein
A⁻ is a counter ion; and
R⁴, R^{4′}, and R⁵ are each independently halogen, C₁₋₁₂ alkyl, or C₂₋₁₂ alkenyl, wherein the alkyl or alkenyl may be substituted with one or more halogen.

27. The method of claim 26, wherein A⁻ is OTf⁻, BF₄⁻, or PF₆⁻.

28. The method of claim 26, wherein the fluorinating agent is a compound having the formula: 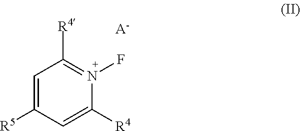
wherein
A⁻ is OTf BF₄⁻, or PF₆⁻;
R⁴ and R^{4′} are each independently a C₁₋₆ alkyl; and
R⁵ is H or a C₁₋₆ alkyl .

29. The method of claim 28 wherein the fluorinating reagent is N-fluoro-2-4-6-trimethylpyridinium triflate.

30. The method of claim 21, wherein the promoter is N-methylpyrrolidinone (NPM).

31. The method of claim 21, wherein the promoter is dimethyl formamide (DMF).

32. The method of claim 21, wherein the yield of the compound of formula (VII) is at least 50%.

33. The method of claim 21, wherein the yield of the compound of formula (VI) is at least 40%.
